(19) United States
(12) Patent Application Publication
(10) Pub. No.: US 2019/0153051 A1
(43) Pub. Date: May 23, 2019

(54) FUSION PROTEIN OF C2 DOMAIN AND AKT KINASE DOMAIN FRAGMENT AND USE THEREOF

(71) Applicants: Gachon University of Industry-Academic Cooperation Foundation, Seongnam-si (KR); Gil Medical Center, Incheon (KR)

(72) Inventors: Byung-Chul Oh, Incheon (KR); Jin Ku Kang, Incheon (KR); Ok Hee Kim, Incheon (KR); Cheol Soon Lee, Incheon (KR)

(21) Appl. No.: 16/198,088

(22) Filed: Nov. 21, 2018

(30) Foreign Application Priority Data
Nov. 22, 2017 (KR) 10-2017-0156848

(51) Int. Cl. C07K 14/47; A61P 3/04; A61P 3/10; A61P 1/16; G01N 33/68

(52) U.S. Cl. CPC C07K 14/4702; A61P 3/04; A61K 38/00; A61P 1/16; G01N 33/68; A61P 3/10

(57) ABSTRACT

The present invention relates to a fusion protein consisting of a C2 domain and an Akt protein fragment, particularly the fragment consisting of the amino acid residues ranging from the 111^{th} to the 480^{th} amino acids from the N-terminus of the Akt protein, and a use of the said fusion protein. More specifically, the fusion protein of the present invention increases the Akt protein activity even under the condition of high calcium concentration, reduces body weight and fat in an animal model treated with a high fat diet infected with adenovirus containing the fusion protein above, improves insulin resistance and improves fatty liver, so that the fusion protein comprising the C2 domain and the fragment consisting of the amino acid residues ranging from the 111^{th} to 480^{th} amino acids from the N-terminus of Akt protein can be effectively used for the treatment of metabolic disease.

### CROSS-REFERENCE TO RELATED APPLICATIONS

This application claims priority to Korean Patent Application Ser. No. 10-2017-0156848 filed Nov. 22, 2017, which is incorporated by reference herein in its entirety.

### BACKGROUND OF THE INVENTION

### 1. Field of the Invention

The present invention relates to a fusion protein consisting of a C2 domain and an Akt kinase domain fragment, particularly the fragment consisting of the amino acid residues ranging from the 111^{th} to the 480^{th} amino acids from the N-terminus of the Akt protein, and use of the said fusion protein.

### 2. Description of the Related Art

Metabolic disease is a disease caused when the metabolism of each organ of our body does not working smoothly. More precisely, metabolic disease is a generalized term for metabolic disorders caused by imbalance of glycosides, lipids, proteins, vitamins, minerals and moisture. In particular, at least 90% of adult disease is attributed to weakened immunity and lack of nutrition.

The representative metabolic diseases include obesity, diabetes, insulin resistance, hyperglycemia, hyperlipidemia, hypercholesterolemia, dyslipidemia and fatty liver. When fat is accumulated in the body due to metabolic diseases, insulin which is a hormone to send blood sugar to the liver or muscle is not properly generated, resulting in insulin resistance with showing reduced insulin function. This causes an increase of blood sugar and atherosclerosis, resulting in the development of adult disease.

One of the metabolic diseases, diabetes has more than 100 million patients worldwide. Diabetes is caused when the blood sugar which is absorbed enough in blood after meals is not absorbed in muscle and fat cells sufficiently. The unabsorbed glucose promotes the synthesis of glycogen, a storage form of glucose, but cannot inhibit the production of new glucose in the liver, which increases the concentration of blood sugar, resulting in hyperglycemia. Chronic diabetes can cause severe diabetic complications.

In the meantime, insulin resistance is caused when there is a mal-functioning in glucose migration and metabolism by insulin in fat cells and skeletal muscle, even though blood insulin concentration is still high. Insulin resistance does not inhibit the production of new glucose in the liver, leading to an increase in blood glucose concentration. Such functional defect is caused by abnormal insulin signaling in the tissues.

In general, the fat accumulated in muscles and liver tissues due to obesity increases intracellular calcium levels, which causes insulin resistance, a major cause of metabolic disorders such as diabetes. According to recent studies, saturated fatty acids inhibited calcium transport in the endoplasmic reticulum (ER) in the obesity or insulin resistance animal model, which resulted in the increase of calcium concentration in the cytoplasm. Such an increase in calcium concentration leads to an adverse effect on the functions of the organs such as ER and mitochondria, resulting in damage in metabolic homeostasis. However, the mechanism how excessive intracellular calcium concentration can cause insulin resistance has not been disclosed, yet.

Korean Patent Publication No. 10-2016-0139072 describes a pharmaceutical composition for treating metabolic disease, specifically obesity and type 2 diabetes, using a scab flower extract or a polyphenolic compound derived therefrom. Korean Patent Publication No. 10-2014-0039322 describes a method for treating metabolic disease such as hyperglycemia using fibroblast growth factor 19 (FGF 19) and fibroblast growth factor 21 (FGF21) having sugar reducing activity.

In the course of study to develop a drug capable of treating metabolic disease, the present inventors constructed a fusion protein of a C2 domain consisting of the amino acid residues ranging from the 159^{th} to the 296^{th} amino acid from the N-terminus of PKCβ (protein kinase C P) and a kinase domain fragment consisting of the amino acid residues ranging from the 111^{th} to the 480^{th} amino acid from the N-terminus of Akt protein (C2-Akt fusion protein). The present inventors further confirmed that the constructed C2-Akt fusion protein was able to increase its activity even under the condition of high calcium concentration induced by obesity and diabetes, reduce body weight and fat in an animal model treated with a high fat diet infected with adenovirus containing the fusion protein above, improve insulin resistance and improves fatty liver, leading to the completion of the present invention.

### SUMMARY OF THE INVENTION

It is an object of the present invention to provide a fusion protein of a C2 domain and an Akt protein fragment, a polynucleotide encoding the fusion protein above, an expression vector containing the polynucleotide above and a host cell transfected with the expression vector above.

It is another object of the present invention to provide a use of the fusion protein of the present invention, the polynucleotide encoding the fusion protein above, the expression vector containing the polynucleotide above and the host cell transfected with the expression vector above.

To achieve the above objects, the present invention provides a fusion protein consisting of a C2 domain and an Akt protein fragment consisting of the amino acid residues ranging from the 111^{th} to 480^{th} amino acid from the N-terminus of Akt protein.

The present invention also provides a polynucleotide encoding the fusion protein above.

The present invention also provides an expression vector containing the polynucleotide above.

The present invention also provides a host cell transfected with the expression vector above.

The present invention also provides a pharmaceutical composition for the prevention or treatment of metabolic disease comprising the fusion protein, the polynucleotide, the expression vector or the host cell of the present invention as an active ingredient.

The present invention also provides a health functional food for the prevention or improvement of metabolic disease comprising the fusion protein, the polynucleotide, the expression vector or the host cell of the present invention as an active ingredient.

The present invention also provides a method for screening a candidate substance for treating metabolic disease comprising the steps of treating the cells expressing the fusion protein of the present invention and having an increased intracellular calcium concentration with a test substance; measuring the binding between the fusion protein of the present invention and calcium in the cells; and selecting a test substance that can inhibit the binding between the fusion protein and calcium.

The present invention further provides a method for screening a candidate substance for treating metabolic disease comprising the steps of treating the mixture consisting of the fusion protein of the present invention and a calcium and PIP complex with a test substance in vitro; measuring the binding between the fusion protein of the invention and the calcium and PIP complex in the mixture above; and selecting a test substance that can inhibit the binding between the fusion protein of the present invention and calcium.

### Advantageous Effect

The fusion protein of the present invention consisting of a C2 domain and an Akt protein fragment comprising the amino acid residues ranging from the 111^{th} to 480^{th} amino acid from the N-terminus of Akt protein increases the Akt protein activity even under the condition of high calcium concentration induced by obesity and diabetes, reduces body weight and fat in an animal model treated with a high fat diet infected with adenovirus containing the fusion protein above, improves insulin resistance and improves fatty liver, so that the fusion protein of the present invention comprising the C2 domain and the fragment consisting of the amino acid residues ranging from the 111^{th} to 480^{th} amino acids from the N-terminus of Akt protein can be effectively used for the treatment of metabolic disease.

### BRIEF DESCRIPTION OF THE DRAWINGS

The application file contains at least one drawing executed in color. Copies of this patent application publication with color drawings will be provided by the Office upon request and payment of the necessary fee.

The application of the preferred embodiments of the present invention is best understood with reference to the accompanying drawings, wherein:

FIG. 1 is a series of diagrams, wherein (A of FIG. 1) is a set of fluorescence microscopic photographs showing that the intracellular calcium concentration in liver tissues of mice fed with high-fat diets (HFD) was increased compared to mice fed with normal diets (chow), (B of FIG. 1) is a graph showing the fluorescence intensity of the photograph, and (C of FIG. 1) is a set of photographs showing the results of Western blotting investigating the activity changes of the proteins involved in insulin signaling.

FIG. 2 is a series of diagrams, wherein (A of FIG. 2) is a set of fluorescence microscopic photographs showing that the intracellular calcium concentration was increased in the insulin resistance induced cells, (B of FIG. 2) is a graph showing the fluorescence intensity of the photograph, and (C of FIG. 2) is a set of photographs showing the results of Western blotting investigating the activity changes of the proteins involved in insulin signaling.

FIG. 3 is a set of graphs illustrating that the intracellular calcium concentration was increased by the treatment of PA in the insulin resistance induced cells.

FIG. 4 illustrates that the Akt protein phosphorylation was inhibited in the cells wherein the calcium concentration was increased by PMA (A of FIG. 4) or ionomycin (B of FIG. 4), while the Akt protein phosphorylation was increased in the cells wherein the calcium concentration was decreased by verapamil (C and D of FIG. 4).

FIG. 5 illustrates that the binding of the PH domain of the purified Akt protein to PI(3,4)P₂ or PI(3,4,5)P₃ was inhibited by increase of the calcium concentration, confirmed by protein-lipid binding assay.

FIG. 6 illustrates that the binding of the PH domain of the purified Akt protein to PI(3,4)P₂ (A of FIG. 6), PI(4,5)P₂ (B of FIG. 6), PI(3,4,5)P₃ (C of FIG. 6) or PS (D of FIG. 6, control) was inhibited by increase of the calcium concentration, confirmed by ITC (isothermal titration calorimetry), and the structures of calcium and PI(3,4,5)P₃ complex and PS (E of FIG. 6).

FIG. 7 is a schematic diagram illustrating that Akt protein PH domain was not combined with PI(3,4,5)P₃ under the condition of increased calcium concentration because calcium was combined with PI(3,4,5)P₃.

FIG. 8 illustrates that the binding of the C2 domain (A of FIG. 8) of PKCβ protein to PI(4,5)P₂ or PI(3,4,5)P₃ was promoted by increase of the calcium concentration, unlike the D187/193A and D246/248A mutant forms (B of FIG. 8) of the C2 domain of PKCβ protein, confirmed by protein-lipid binding assay.

FIG. 9 illustrates that the C2 domain (B of FIG. 9) of PKCβ protein was combined with PI(3,4,5)P₃ under the condition of increased calcium concentration, but the binding was not induced in the absence of calcium (A of FIG. 9), while the C2 domain mutant (C of FIG. 9) was not combined with PI(3,4,5)P₃, confirmed by ITC.

FIG. 10 is a set of photographs illustrating that the migration of the PH domain (A of FIG. 10) of Akt protein) in the cell membrane was inhibited by the increased calcium concentration, while the migration of the C2 domain (B of FIG. 10) in the cell membrane was accelerated by the increased calcium concentration.

FIG. 11 shows that the activity of the wild-type Atk protein (A of FIG. 11) was inhibited under the condition of increased calcium concentration induced by the treatment of PMA, confirmed by Western blotting (B of FIG. 11), and the expression changes of pAkt(T308) (C of FIG. 11) and pAkt(T473) (D of FIG. 11) proteins; and that the Akt protein activity was increased by the C2-Akt fusion protein (E of FIG. 12) under the condition of increased calcium concentration, confirmed by Western blotting (F of FIG. 11), and the expression changes of pAkt(T308) (G of FIG. 11) and pAkt(T473) (H of FIG. 11) proteins.

FIG. 12 shows that the activity of the wild-type Atk protein was inhibited under the condition of increased calcium concentration induced by the treatment of PMA, confirmed by Western blotting (A of FIG. 12), and the expression changes of pAkt(T308) (B of FIG. 12) and pAkt(T473) (C of FIG. 12) proteins; and that the Akt protein activity was increased by the C2-Akt fusion protein under the condition of increased calcium concentration, confirmed by Western blotting (D of FIG. 12), and the expression changes of pAkt(T308) (E of FIG. 12) and pAkt(T473) (F of FIG. 12) proteins.

FIG. 13 shows the results of confirming the protein expression by treating PTEN (A of FIG. 13) or SHIP2 (B of FIG. 13), which was performed to determine whether PI(4,5)P₂ or PI(3,4,5)P₃ was more important in the signal transduction mechanism by the C2-Akt fusion protein.

FIG. 14 presents a schematic diagram (A of FIG. 14) illustrating the fusion protein prepared by using a mutant form of the C2 domain in the C2-Akt fusion protein; a diagram (B of FIG. 14) illustrating the results of Western blotting performed to identify an amino acid residue in the C2 domain that was able to increase the Akt protein activity by using the fusion protein above; and a graph (C of FIG. 14) illustrating the results of Western blotting above.

FIG. 15 is a set of schematic diagrams illustrating the structure of the adenovirus vector (A of FIG. 15) inserted in the animal model fed with high fat diet and the animal experiment plan (B of FIG. 15).

FIG. 16 is a set of graphs illustrating the weight loss (A of FIG. 16) and the changes in feed intake (B of FIG. 16) according to the treatment of the C2-Akt fusion protein in the animal model fed with high fat diet.

FIG. 17 is a set of graphs illustrating the fat reduction (A of FIG. 17) and the reduction of normal weight obesity (B of FIG. 17) by the C2-Akt fusion protein in the animal model fed with high fat diet.

FIG. 18 is a set of graphs illustrating the reduction of glucose levels (A and B of FIG. 18), the increase of insulin sensitivity (C of FIG. 18) and the decrease in fasting insulin levels (D of FIG. 18) according to the treatment of the C2-Akt fusion protein in the animal model fed with high fat diet.

FIG. 19 is a set of graphs illustrating the changes in fasting glucose (A of FIG. 19) and insulin (B of FIG. 19) levels in the animal model fed with high fat diet and the HOMA-IR (C of FIG. 19) calculated from the above values.

FIG. 20 is a set of graphs illustrating the ALT (A of FIG. 20), total cholesterol (B of FIG. 20) and LDL (C of FIG. 20) levels measured in the blood sample of the animal model fed with high fat diet.

FIG. 21 is a set of photographs illustrating the liver tissue (A of FIG. 21) of the animal model fed with high fat diet according to the treatment of the C2-Akt fusion protein and the reduction of lipid droplets in the liver tissue above (B of FIG. 21).

FIG. 22 is a set of photographs illustrating the results of Western blotting performed to confirm the Akt activity changes in the liver tissue of the high fat diet animal model treated with the C2-Akt fusion protein.

FIG. 23 is a set of graphs and photographs illustrating the expression changes of the genes involved in glucose production (A of FIG. 23), fat production (B of FIG. 23) and fat absorption (C of FIG. 23) in the liver tissue of the high fat diet animal model treated with the C2-Akt fusion protein and the expression changes (D of FIG. 23) of the proteins expressed by the genes.

FIG. 24 shows 5 types of genes up-regulated by the C2-Akt fusion protein and 7 types of genes (A of FIG. 24) down-regulated by the C2-Akt fusion protein; and the expression changes of the genes up-regulated above (B of FIG. 24), the genes down-regulated above (C of FIG. 24) and the genes that promote fatty liver (D of FIG. 24).

### DESCRIPTION OF THE PREFERRED EMBODIMENTS

Hereinafter, the present invention is described in detail.

The present invention provides a fusion protein comprising a C2 domain and an Akt protein fragment consisting of the amino acid residues ranging from the 111^{th} to 480^{th} amino acid from the N-terminus of Akt protein.

The term “C2 domain” used in this invention indicates a protein structural domain involved in targeting a protein to the cell membrane.

The C2 domain generally has a calcium dependence which binds to 2 to 3 calcium ions and a calcium independence which does not require calcium ions. The C2 domain is generally either calcium dependent which is combined with 2-3 calcium ions or calcium independent which does not need calcium ions. The C2 domain has a β-sandwich consisting of 8 β-strands that regulate calcium ions and the cavity formed by the first and last loops thereof is fused with phospholipids of the cell membrane.

The C2 domain is found in about 362 kinds of proteins, and 582 of them are known to exist (http://smart.embl.de/smart/do_annotation.pl?DOMAIN=SM00239). Particularly, the C2 domain can be originated from PI3K (phosphatidylinositol 3-kinase), PKC (protein kinase C), ABR (active breakpoint cluster region-related), BAIAP3 (BAI1-associated protein 3), BCR (breakpoint cluster region), C2CD2 (C2 calcium dependent domain containing 2), C2CD3 (C2 calcium dependent domain containing 3), CADPS (calcium-dependent secretion activator 1), CADPS2 (calcium-dependent secretion activator 2), CAPN5 (calpain-5), CAPN6 (calpain-6), CC2D1A (coiled-coil and C2 domain-containing protein 1A), CC2D1B (coiled-coil and C2 domain-containing protein 1B), CPNE1 (copine-1), CPNE2 (copine-2), CPNE3 (copine-3), CPNE4 (copine-4), CPNE5 (copine-5), CPNE6 (copine-6), CPNE7 (copine-7), CPNE8 (copine-8), CPNE9 (copine-9), DAB2IP (disabled homolog 2-interacting protein), DOC2A (double C2-like domain-containing protein alpha), DOC2B (double C2-like domain-containing protein beta), DYSF (dysferlin), ESYT1 (extended synaptotagmin-1), ESYT3 (extended synaptotagmin-3), FAM62B (extended synaptotagmin-2), FER1L3 (myoferlin), FER1L5 (fer-1 like family member 5), HECW1 (C2 and WW domain containing E3 ubiquitin protein ligase 1), HECW2 (C2 and WW domain containing E3 ubiquitin protein ligase 1), ITCH (itchy E3 ubiquitin protein ligase), ITSN1 (intersectin-1), ITSN2 (intersectin-2), MCTP1 (multiple C2 and transmembrane domain containing 1), MCTP2 (multiple C2 and transmembrane domain containing 2), MTAC2D1 (tandem C2 domains nuclear protein), NEDD4 (neural precursor cell expressed developmentally down-regulated protein 4), NEDD4L (neural precursor cell expressed developmentally down-regulated gene 4-like), OTOF (otoferlin), PCLO (protein piccolo), PIK3C2A (phosphatidylinositol-4-phosphate 3-kinase C2 domain-containing alpha), PIK3C2B (phosphatidylinositol-4-phosphate 3-kinase C2 domain-containing beta), PIK3C2G (phosphatidylinositol-4-phosphate 3-kinase C2 domain-containing gamma), PLA2G4A (cytosolic phospholipase A2), PLA2G4B (cytosolic phospholipase A2 beta), PLA2G4D (cytosolic phospholipase A2 delta), PLA2G4E (cytosolic phospholipase A2 epsilon), PLA2G4F (cytosolic phospholipase A2 zeta), PLCB1 (1-phosphatidylinositol-4,5-bisphosphate phosphodiesterase beta-1), PLCB2 (1-phosphatidylinositol-4,5-bisphosphate phosphodiesterase beta-2), PLCB3 (1-phosphatidylinositol-4,5-bisphosphate phosphodiesterase beta-3), PLCB4 (1-phosphatidylinositol-4,5-bisphosphate phosphodiesterase beta-4), PLCD1 (phospholipase C delta 1), PLCD3 (phospholipase C delta 3), PLCD4 (phospholipase C delta 4), PLCE1 (phospholipase C epsilon 1), PLCG1 (phospholipase C gamma 1), PLCG2 (phospholipase C gamma 2), PLCH1 (phospholipase C eta 1), PLCH2 (phospholipase C eta 2), PLCL1 (phospholipase C like 1), PLCL2 (phospholipase C like 2), PLCZ1 (phospholipase C zeta 1), PRF1 (perforin-1), PRKCA (protein kinase C alpha), PRKCB1 (protein kinase C beta type), PRKCE (protein kinase C epsilon), PRKCG (protein kinase C gamma), PRKCH (protein kinase C eta), RAB11FIP1 (Rab11 family-interacting protein 1), RAB11FIP2 (Rab11 family-interacting protein 2), RAB11FIP5 (Rab11 family-interacting protein 5), RASA1 (RAS p21 protein activator 1), RASA2 (RAS p21 protein activator 2), RASA3 (RAS p21 protein activator 3), RASA4 (RAS p21 protein activator 4), RASAL1 (RAS protein activator like 1), RASAL2 (RAS protein activator like 2), RGS3 (regulator of G-protein signaling 3), RIMS1 (regulating synaptic membrane exocytosis protein 1), RIMS2 (regulating synaptic membrane exocytosis protein 2), RIMS3 (regulating synaptic membrane exocytosis protein 3), RIMS4 (regulating synaptic membrane exocytosis protein 4), RPGRIP1 (X-linked retinitis pigmentosa GTPase regulator-interacting protein 1), RPH3A (rabphilin-3A), SMURF1 (E3 ubiquitin-protein ligase SMURF1), SMURF2 (E3 ubiquitin-protein ligase SMURF2), SYNGAP1 (synaptic Ras GTPase-activating protein 1), SYT1 (synaptotagmin-1), SYT10 (synaptotagmin-10), SYT11 (synaptotagmin-11), SYT12 (synaptotagmin-12), SYT13 (synaptotagmin-13), SYT14 (synaptotagmin-14), SYT15 (synaptotagmin-15), SYT16 (synaptotagmin-16), SYT17 (synaptotagmin-17), SYT2 (synaptotagmin-2), SYT3 (synaptotagmin-3), SYT4 (synaptotagmin-4), SYT5 (synaptotagmin-5), SYT6 (synaptotagmin-6), SYT7 (synaptotagmin-7), SYT8 (synaptotagmin-8), SYT9 (synaptotagmin-9), SYTL1 (synaptotagmin like 1), SYTL2 (synaptotagmin like 2), SYTL3 (synaptotagmin like 3), SYTL4 (synaptotagmin like 4), SYTL5 (synaptotagmin like 5), TOLLIP (toll interacting protein), UNC13A (Unc-13 homolog A), UNC13B (Unc-13 homolog B), UNC13C (Unc-13 homolog C), UNC13D (Unc-13 homolog D), WWC2 (WW and C2 domain containing 2), WWP1 (WW domain containing E3 ubiquitin protein ligase 1), WWP2 (WW domain containing E3 ubiquitin protein ligase 2) or PTEN (phosphatase and tensin homolog) protein.

In addition, the C2 domain can be a polypeptide consisting of any sequence known to those in this art. The polypeptide can include a mutant amino acid having different sequences or a fragment thereof produced by deletion, insertion and substitution of amino acid residues or a combination thereof, as long as such modification does not affect the protein function. Amino acid exchange in proteins or peptides that do not generally alter the activity of the molecule is well informed to those in the art. The polypeptide can be modified by phosphorylation, sulfation, acrylation, glycosylation, methylation, and farnesylation, etc. In a preferred embodiment of the present invention, the C2 domain can be a polypeptide consisting of the amino acid sequence represented by SEQ. ID NO: 14.

The term “Akt (protein kinase B) protein” used in this invention is a protein that plays an important role in the insulin signaling system mediated by PI(3,4)P₂ or PI(3,4,5)P₃. In particular, the Akt protein mediates downstream signal transduction by phosphorylating various proteins such as GSK3β (glycogen synthase kinase 3β), AS160 (160 kDa substrate of Akt), and FOXO3 (the fork head transcription factor). The Akt protein has a pleckstrin homology (PH) domain consisting of approximately 120 amino acids at its N-terminus. This domain interacts directly with the cell membrane by binding to phosphatidylinositol phosphate (PIP).

The Akt protein can be derived from a mammal, particularly a human, a rat, a rabbit, a mouse, a sheep, a dog, and a pig, etc. In addition, the Akt protein can be a polypeptide consisting of any sequence known to those in the art, and can include a variant or a fragment thereof having the above-mentioned characteristics. In a preferred embodiment of the present invention, the Akt protein can be a polypeptide consisting of the amino acid sequence represented by SEQ. ID. NO: 16. In the meantime, the PH domain of Akt protein can be a polypeptide consisting of the amino acid sequence represented by SEQ. ID. NO: 13.

The fusion protein of the present invention can be a fusion protein comprising a fragment consisting of the amino acid residues ranging from the 111^{th} to 480^{th} amino acid from the N-terminus of Akt protein. The fusion protein can be obtained by replacing the PH domain of Akt protein with the C2 domain. The fusion protein can be constructed by using a proper sequence by those in the art, which can also contain a mutant or a fragment having the above-mentioned characteristics. In a preferred embodiment of the present invention, the fusion protein can be a polypeptide consisting of the amino acid sequence represented by SEQ. ID. NO: 17.

The fusion protein above can additionally include a fluorescent protein. The fluorescent protein above can be combined to the C2 domain of the present invention and any one selected from the group consisting of the N-terminus and the C-terminus of the fusion protein fused with a fragment consisting of the amino acid residues ranging from the 111^{th} to 480^{th} amino acid from the N-terminus of Akt protein. The fluorescent protein can include any protein well informed to those in the art, which is preferably GFP, YFP or mCherry protein.

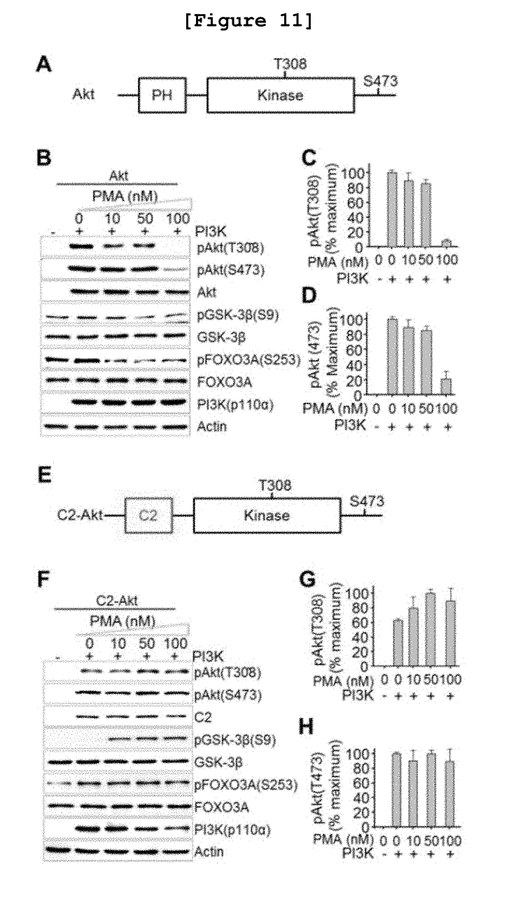

In a preferred embodiment of the present invention, the present inventors obtained a polynucleotide encoding the Akt protein other than the C2 domain and the PH domain of PKCβ protein through PCR, and cloned it into an expression vector to construct a fusion protein in which the C2 domain was fused instead of the PH domain of Akt protein (see FIG. 11).

The present invention also provides a polynucleotide encoding the fusion protein of the present invention.

The fusion protein of the present invention can have the above-mentioned characteristics. As an example, the fusion protein above can be such a fusion protein that is consisting of a C2 domain and an Akt protein fragment comprising the amino acid residues ranging from the 111^{th} to 480^{th} amino acid from the N-terminus of Akt protein. The C2 domain can be originated from any protein well known to those in the art, and preferably can be a polypeptide consisting of the amino acid sequence represented by SEQ. ID. NO: 14.

Meanwhile, the fragment of the Akt protein may be a fragment in which, specifically, a polypeptide consisting of the amino acid sequence of SEQ ID NO: 15. In the meantime, the Akt protein fragment can be a fragment in which the PH domain of Akt protein is deleted, and preferably a polypeptide consisting of the amino acid sequence represented by SEQ. ID. NO: 15.

The polynucleotide can include any sequence as long as it is a polynucleotide encoding the fusion protein of the present invention. Particularly, the polynucleotide can be a polynucleotide consisting of the nucleotide sequence represented by SEQ. ID. NO: 18. The polynucleotide above can have homology as high as 70, 80, 90 or 95% with the nucleotide sequence represented by SEQ. ID. NO: 18.

The present invention also provides an expression vector containing the polynucleotide of the present invention.

The expression vector can be any vector capable of expressing the fusion protein of the present invention in a desired host cell. The term “vector” herein indicates a recombinant vector that can express a target peptide in a host cell, precisely a gene construct containing a necessary regulatory element operably linked to the construct in order to express a gene insert. The vector above includes expression regulatory elements such as an initiation codon, a termination codon, a promoter and an operator. The initiation codon and the termination codon are generally considered as a partial sequence of a nucleotide encoding a polypeptide, which are supposed to be functioning well when a gene construct is inserted and have to be located in frame of a coding sequence.

The present invention also provides a host cell containing the expression vector of the present invention.

The host cell is a cell transfected with an expression vector containing a polynucleotide according to the present invention, which is preferably a mammalian cell herein. The transfection can be performed by the conventional method well informed to those in the art. For example, physical methods such as microinjection, liposome-based methods, directed DNA uptake, receptor-mediated DNA transfer, Ca⁺⁺-mediated DNA transfer, or virus mediated gene transfer can be used.

In the meantime, the mammalian cell above can be NS/0 myeloma cell, 293 cell, CHO cell, HeLa cell, CapT cell (human amniotic fluid derived cell) or COS cell.

The present invention also provides a pharmaceutical composition for the prevention or treatment of metabolic disease comprising the fusion protein, the polynucleotide, the expression vector or the host cell of the present invention as an active ingredient.

The fusion protein, the polynucleotide, the expression vector or the host cell of the present invention can have the above-mentioned characteristics. As an example, the fusion protein above can be such a fusion protein that is consisting of a C2 domain and an Akt protein fragment comprising the amino acid residues ranging from the 111^{th} to 480^{th} amino acid from the N-terminus of Akt protein. The C2 domain can be originated from any protein well known to those in the art, and preferably can be a polypeptide consisting of the amino acid sequence represented by SEQ. ID. NO: 14. In the meantime, the Akt protein fragment can be a fragment in which the PH domain of Akt protein is deleted, and preferably a polypeptide consisting of the amino acid sequence represented by SEQ. ID. NO: 15.

The metabolic disease can be obesity, insulin resistance, diabetes or fatty liver.

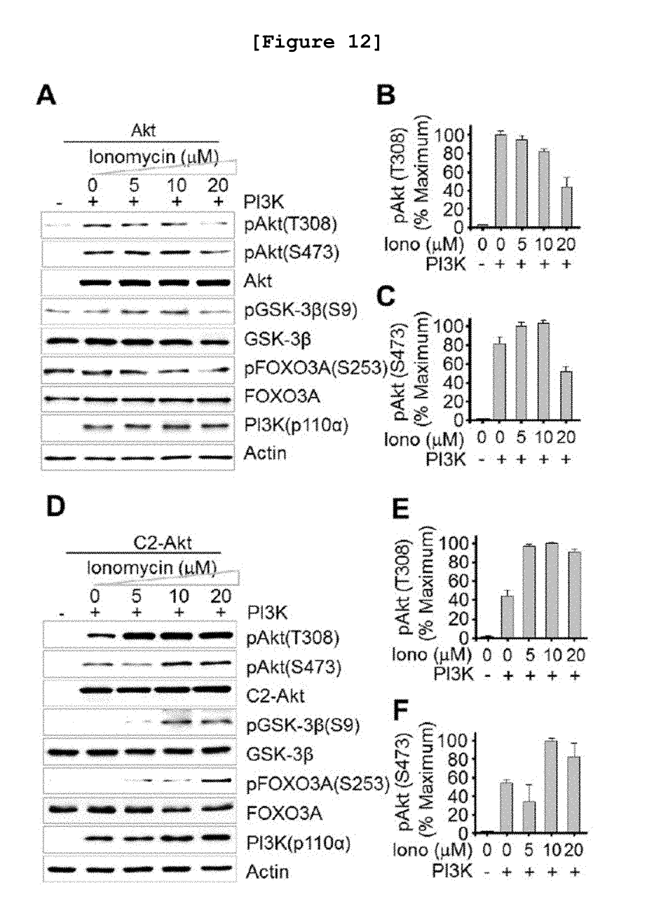

In a preferred embodiment of the present invention, the present inventors constructed a C2-Akt fusion protein fused with the C2 domain instead of the PH domain of Akt protein and transfected the C2-Akt fusion protein into HepG2 cells. The Akt protein activity in the cells transfected with the C2-Akt fusion protein of the present invention was activated even when the concentration of calcium was increased (see FIG. 11 and FIG. 12).

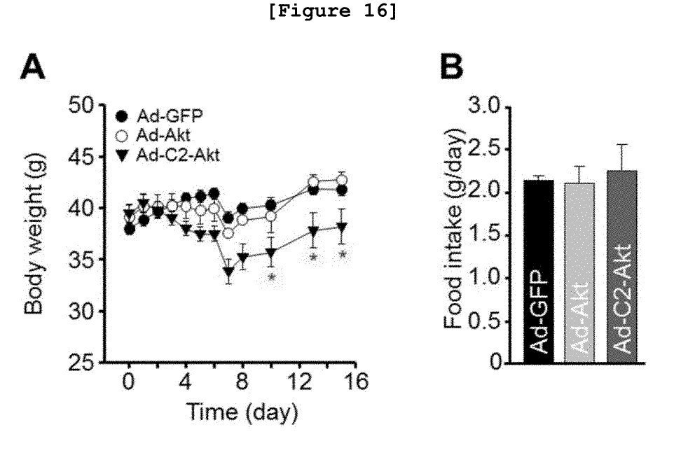

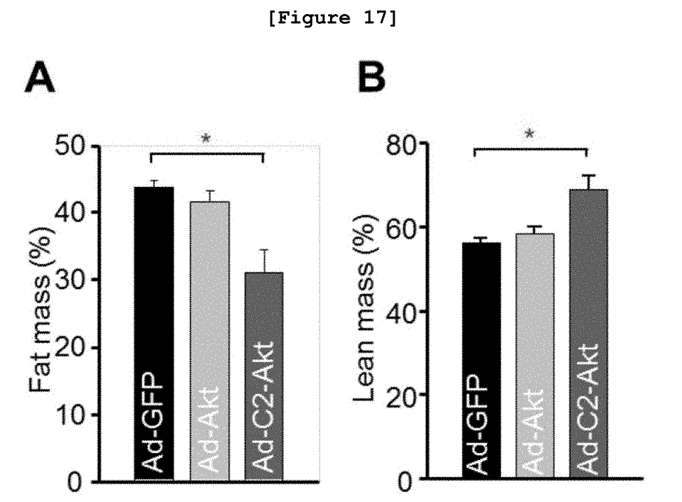

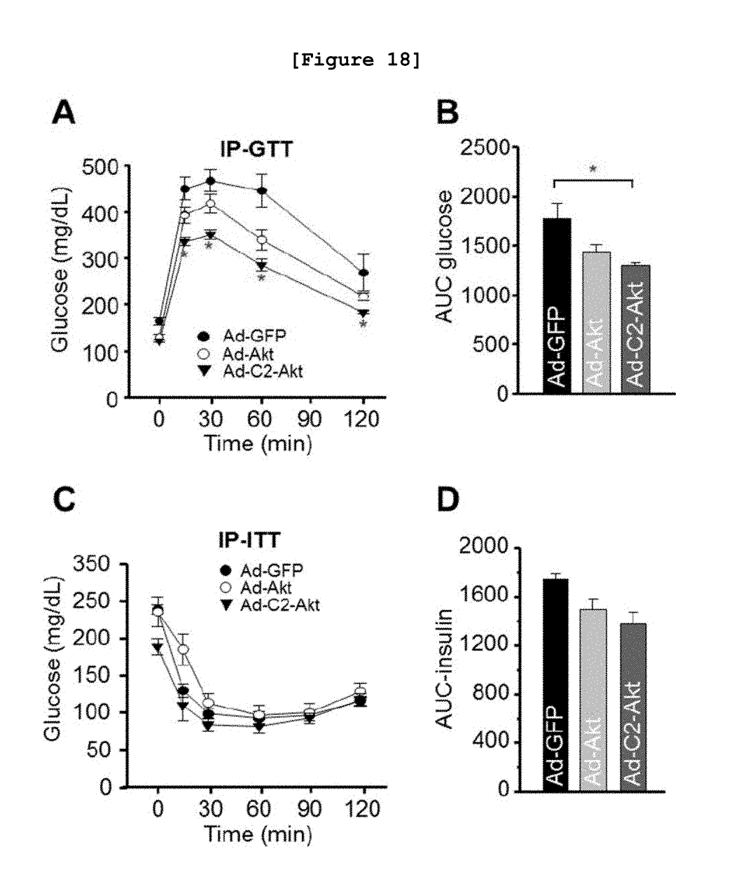

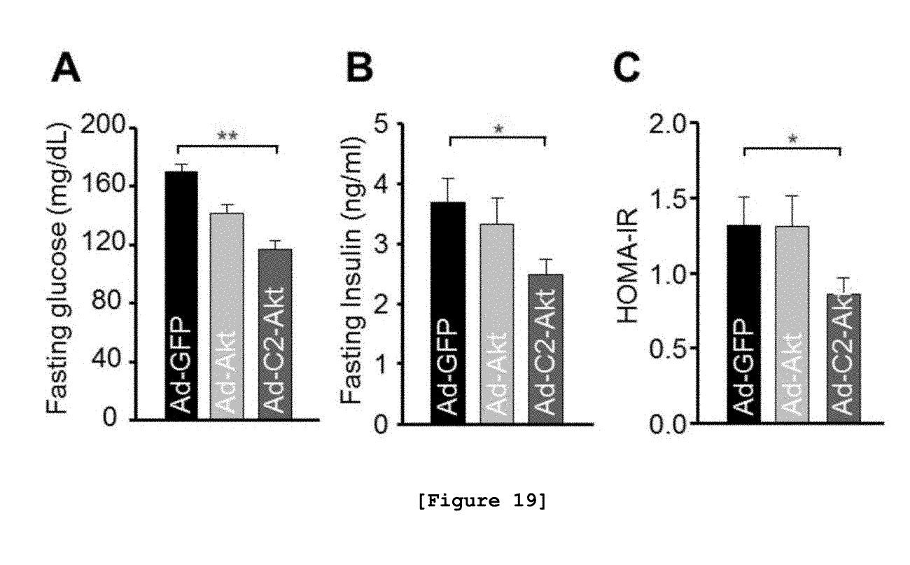

The high fat diet mouse model was infected with the adenovirus vector expressing the C2-Akt fusion protein. As a result, the body weight, fat mass and dry weight of the mouse were reduced by the fusion protein of the present invention (see FIG. 16 and FIG. 17). The mouse animal model has decreased glucose concentration in the body by the C2-Akt fusion protein and improved insulin resistance. The in vivo glucose level was reduced and the insulin resistance was improved in the mouse model by the C2-Akt fusion protein (see FIG. 18 and FIG. 19).

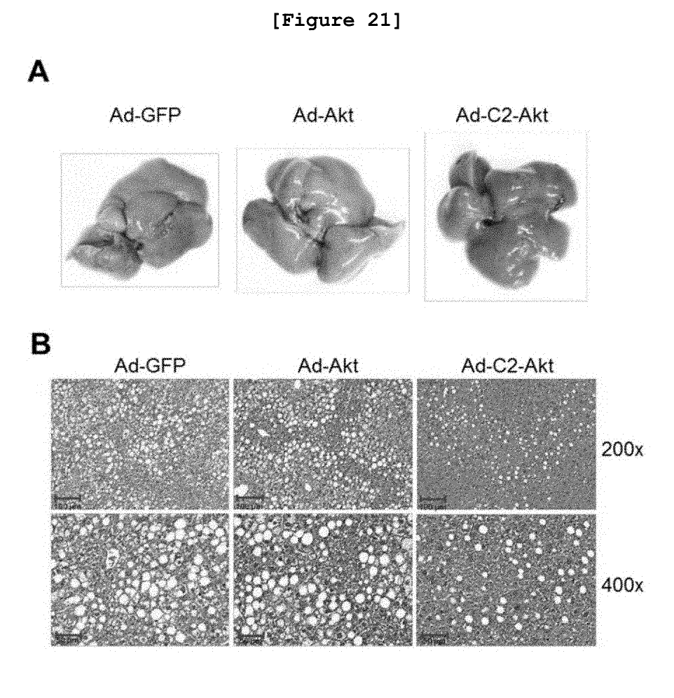

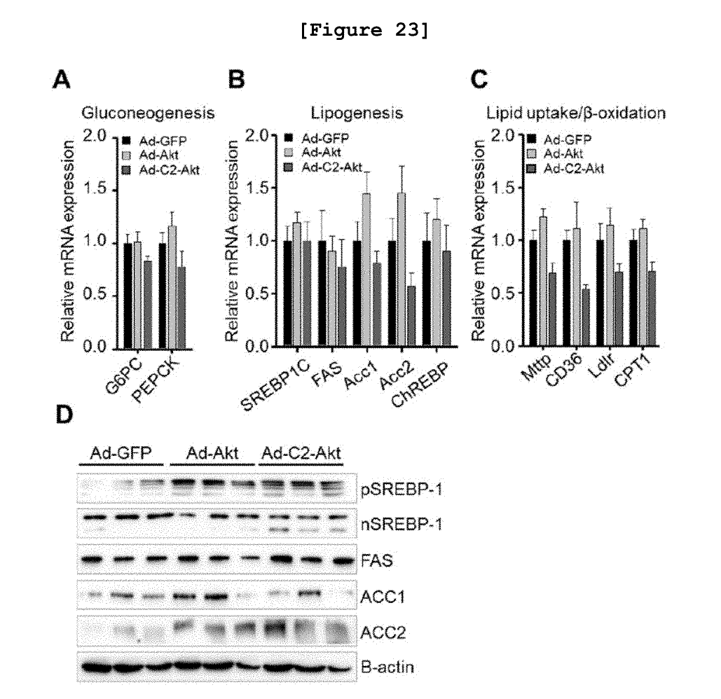

The present inventors investigated whether or not the fatty liver induced in the mouse model was improved by the C2-Akt fusion protein. As a result, it was confirmed that the C2-Akt fusion protein reduced lipid droplets (FIG. 21), reduced the expression of such gene that was involved in glucose and fat synthesis or fat absorption, and also reduced the protein expression induced by the gene (see FIG. 23).

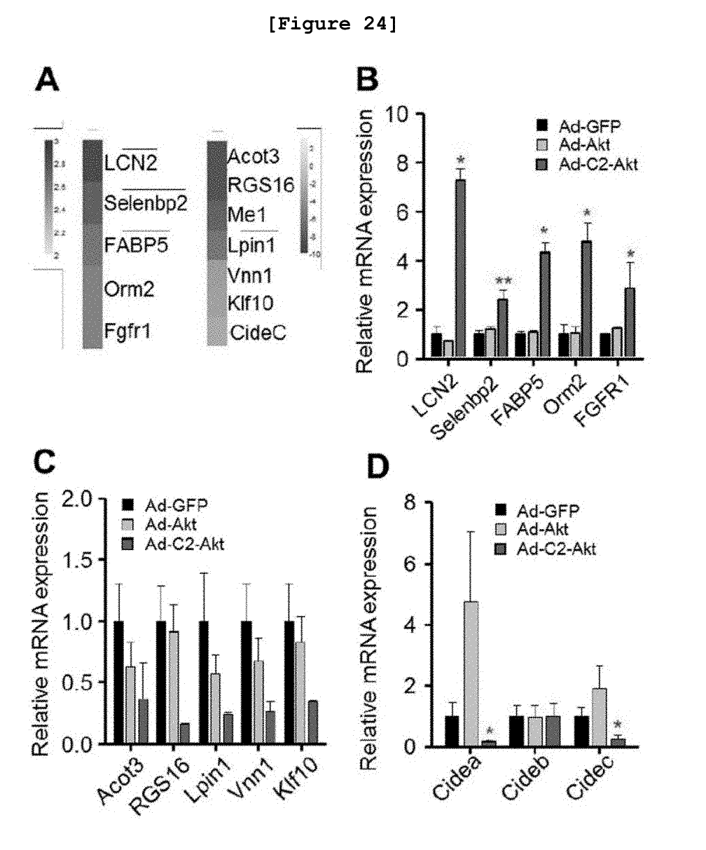

Further, the present inventors investigated the expression patterns of genes and proteins that improve fatty liver or promote fatty liver in the mouse model. As a result, it was confirmed that the C2-Akt fusion protein increased the expressions of such genes and proteins that improved fatty liver, but inhibited the expressions of such genes and proteins that promoted fatty liver. In addition, the C2-Akt fusion protein also inhibited the expressions of such genes involved in lipid droplets formation and fat storage (see FIG. 24).

Therefore, the C2-Akt fusion protein of the present invention can be effectively used for the treatment of such metabolic diseases as obesity, insulin resistance, diabetes, and fatty liver, etc.

The pharmaceutical composition of the present invention can contain the fusion protein, the polynucleotide, the expression vector or the host cell of the present invention as an active ingredient at the concentration of 10 to 95 weight %. In addition to the active ingredient above, the pharmaceutical composition can additionally contain one or more active ingredients showing the same or a similar function to the active ingredient above.

The pharmaceutical composition of the present invention can include any generally used carriers, diluents, excipient, or a combination of at least two of those. The pharmaceutically acceptable carrier can be any carrier that is able to deliver the composition of the present invention in living body without limitation, which is exemplified by the compounds described in Merck Index, 13^{th} ed., Merck & Co. Inc., such as saline, sterilized water, Ringer's solution, buffered saline, dextrose solution, maltodextrin solution, glycerol, ethanol, or a mixture comprising one or more of those components. If necessary, a general additive such as antioxidant, buffer, and bacteriostatic agent can be additionally added.

The pharmaceutical composition of the present invention can be formulated by using generally used diluents or excipients such as fillers, extenders, binders, wetting agents, disintegrating agents and surfactants.

The pharmaceutical composition of the present invention can be formulated into an oral preparation or a parenteral preparation. Solid formulations for oral administration are tablets, pills, powders, granules, capsules and troches. These solid formulations are prepared by mixing the said composition with one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives.

Formulations for parenteral administration can include injections such as sterilized aqueous solutions, water-insoluble solutions, suspensions, and emulsions. As the water insoluble solutions and suspensions, propylene glycol, polyethylene glycol, vegetable oil like olive oil, and injectable ester like ethylolate can be used.

The pharmaceutical composition of the present invention can be administered orally or parenterally according to the desired method and the parenteral administration includes topical application, intraperitoneal injection, intrathecal injection, subcutaneous injection, intravenous injection, intramuscular injection or intrathoracic injection.

The pharmaceutical composition of the present invention can be administered in a pharmaceutically effective dose. The effective dose can be determined according to the type of disease, severity, activity of the drug, sensitivity to the drug, administration time, administration pathway, rate of excretion, duration of treatment, and the drug used simultaneously. The composition of the present invention can be administered alone or in combination with other therapeutic agents. The composition of the present invention can be administered sequentially or simultaneously when administered concurrently.

For a desired effect, the amount of the active ingredient contained in the pharmaceutical composition of the present invention can be 0.001 to 10,000 mg/kg, specifically 0.1 to 5 g/kg. The administration frequency is once a day or a few times a day.

The present invention also provides a health functional food for the prevention or improvement of metabolic disease comprising the fusion protein, the polynucleotide, the expression vector or the host cell of the present invention as an active ingredient.

The fusion protein, the polynucleotide, the expression vector or the host cell of the present invention can have the above-mentioned characteristics. As an example, the fusion protein above can be such a fusion protein that is consisting of a C2 domain and an Akt protein fragment comprising the amino acid residues ranging from the 111^{th} to 480^{th} amino acid from the N-terminus of Akt protein. The C2 domain can be originated from any protein well known to those in the art, and preferably can be a polypeptide consisting of the amino acid sequence represented by SEQ. ID. NO: 14. In the meantime, the Akt protein fragment can be a fragment in which the PH domain of Akt protein is deleted, and preferably a polypeptide consisting of the amino acid sequence represented by SEQ. ID. NO: 15.

The metabolic disease can be obesity, insulin resistance, diabetes or fatty liver.

In a preferred embodiment of the present invention, the present inventors constructed a C2-Akt fusion protein by combining the C2 domain and the PH domain deleted Akt protein and further confirmed that the constructed fusion protein above was able to maintain the Akt protein activity under the condition of high calcium concentration (see FIG. 11 and FIG. 12).

The C2-Akt fusion protein above was confirmed to improve such symptoms as obesity, insulin resistance, diabetes and fatty liver in the mouse model fed with high fat diet (see FIGS. 16-19, 23, and 24), indicating that the C2-Akt fusion protein of the present invention can be effectively used for the improvement of metabolic disease.

The composition of the present invention can be added directly to food or can be used with other food or food ingredients. At this time, the content of the active ingredient to be added can be determined depending on the purpose, and can generally be 0.01 to 90 weight parts by the total food weight.

The form and the kind of the health functional food are not particularly limited. Particularly, the health functional food can be in the form of tablets, capsules, powders, granules, liquid preparations and pills. The health functional food can contain various flavors, sweeteners or natural carbohydrates as an additional ingredient. The sweeteners can be natural or synthetic sweeteners, examples of natural sweeteners include thaumatin and stevia extract. On the other hand, examples of synthetic sweeteners include saccharin and aspartame. The natural carbohydrates can be monosaccharides, disaccharides, polysaccharides, oligosaccharides, and sugar alcohols, etc.

In addition to the ingredients mentioned above, the health functional food of the present invention can include in a variety of nutrients, vitamins, minerals, flavors, coloring agents, pectic acid and its salts, alginic acid and its salts, organic acid, protective colloidal viscosifiers, pH regulators, stabilizers, antiseptics, glycerin, and alcohols, etc. All the mentioned ingredients can be added singly or together. The mixing ratio of those ingredients does not matter in fact, but in general, each can be added by 001 to 0.1 weight part per 100 weight part of the composition of the present invention.

The present invention also provides a method for screening a candidate substance for treating metabolic disease comprising the steps of treating the cells expressing the fusion protein of the present invention and having an increased intracellular calcium concentration with a test substance; measuring the binding between the fusion protein of the present invention and calcium in the cells; and selecting a test substance that can inhibit the binding between the fusion protein and calcium.

The said calcium can be combined with phosphatidyl inositol (3,4,5)-triphosphate or phosphatidyl inositol (4,5)-bisphosphate. In the screening method of the present invention, the binding between the fusion protein of the present invention and calcium can be measured by any conventional method well known to those in the art, which is preferably selected from the group consisting of ITC method and protein-lipid binding assay.

In the screening method of the present invention, the cells showing a higher calcium concentration above can be an obesity cell model, an insulin resistance cell model, a diabetes cell model or a fatty liver cell model, and the candidate substance screened by using the cell model above can be the candidate for the drug to treat metabolic disease, preferably obesity, insulin resistance, diabetes or fatty liver.

The screening method of the present invention can include an additional step of selecting a test substance that can inhibit the migration of the fusion protein of the present invention to the cell membrane or inhibit the Akt signal transduction system by the fragment consisting of the amino acid residues ranging from the 111^{th} amino acid to the 480^{th} amino acid from the N-terminus of Akt protein included in the fusion protein above.

Further, the fusion protein of the present invention can additionally include a detection reagent or a fluorescent protein. The detection reagent herein can be a conjugate labeled with a detection maker such as chromogenic enzyme, a fluorescent material, a radioactive isotope or colloid. The said chromogenic enzyme can be peroxidase, phosphatase or acid phosphatase.

The fluorescent material herein can be fluorescein carboxylic acid (FCA), fluorescein isothiocyanate (FITC), fluorescein thiourea (FTH), 7-acetoxycoumarin-3-yl, fluorescein-5-yl, fluorescein-6-yl, 2′,7′-dichlorofluorescein-5-yl, 2′,7′-dichlorofluorescein-5-yl, dihydrotetramethylrosamin-4-yl, tetramethyl rhodamine-5-yl, tetramethyl rhodamine-6-yl, 4,4-difluoro-5,7-dimethyl-4-bora-3a, 4a-diaza-s-indacene-3-ethyl or 4,4-difluoro-5,7-diphenyl-4-bora-3a, 4a-diaza-s-indacene-3-ethyl, Cy3, Cy5, poly-L-lysine-fluorescein isothiocyanate (FITC), rhodamnine-B-isothiocyanate (RITC), PE (Phycoerythrin) or rhodamine. The said fluorescent protein can bind to one or more terminals selected from the group consisting of the N-terminal and the C-terminal of the fusion protein of the present invention. The fluorescent protein herein can include any protein well informed to those in the art, which is preferably exemplified by GFP, YFP or mCherry.

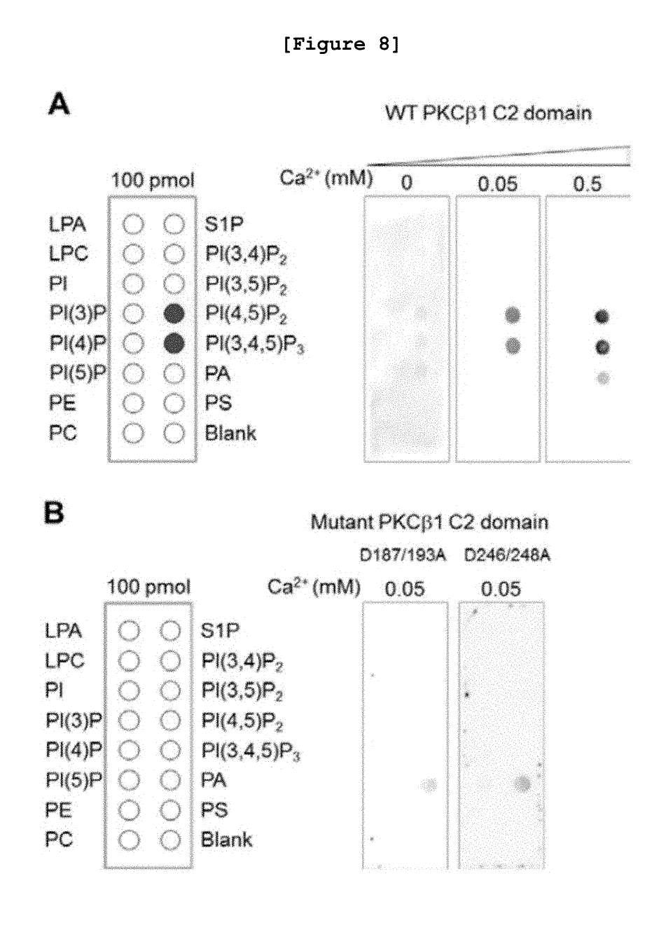

In a preferred embodiment of the present invention, the present inventors confirmed that the calcium concentration was increased in the mouse model fed with high fat diet and the cells induced with insulin resistance and the increased calcium interacted with the C2 domain to bind PIP, which migrated to the cell membrane (see FIG. 8 to FIG. 10).

Therefore, from the above results, it was confirmed that the fusion protein of the present invention containing the C2 domain can be effectively used for screening a candidate substance for treating metabolic disease by using the process of selecting a substance that was able to inhibit the fusion between calcium and the PIP complex in cells in which intracellular calcium concentration is increased.

The present invention further provides a method for screening a candidate substance for treating metabolic disease comprising the steps of treating the mixture consisting of the fusion protein of the present invention and a calcium and PIP complex with a test substance in vitro; measuring the binding between the fusion protein of the present invention and the calcium and PIP complex in the mixture above; and selecting a test substance that can inhibit the binding between the fusion protein of the present invention and calcium.

The said calcium can be combined with phosphatidyl inositol (3,4,5)-triphosphate or phosphatidyl inositol (4,5)-bisphosphate. In the screening method of the present invention, the binding between the fusion protein of the present invention and calcium can be measured by any conventional method well known to those in the art, which is preferably selected from the group consisting of ITC method and protein-lipid binding assay.

The metabolic disease herein can be obesity, insulin resistance, diabetes or fatty liver.

In a preferred embodiment of the present invention, the present inventors confirmed that the calcium concentration was increased in the mouse model fed with high fat diet and the cells induced with insulin resistance and the increased calcium interacted with the C2 domain to bind PIP, which migrated to the cell membrane (see FIG. 8 to FIG. 10). Therefore, from the above results, it was confirmed that the fusion protein of the present invention containing the C2 domain can be effectively used for screening a candidate substance for treating metabolic disease by using the process of selecting a substance that was able to inhibit the fusion between the fusion protein of the present invention and the calcium and PIP complex.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1: Confirmation of Increased Calcium Level in Insulin Resistance Animal Model Induced by Obesity

The following experiment was performed in order to confirm the changes of intracellular calcium concentration in liver cells of the insulin resistance animal model induced by obesity.

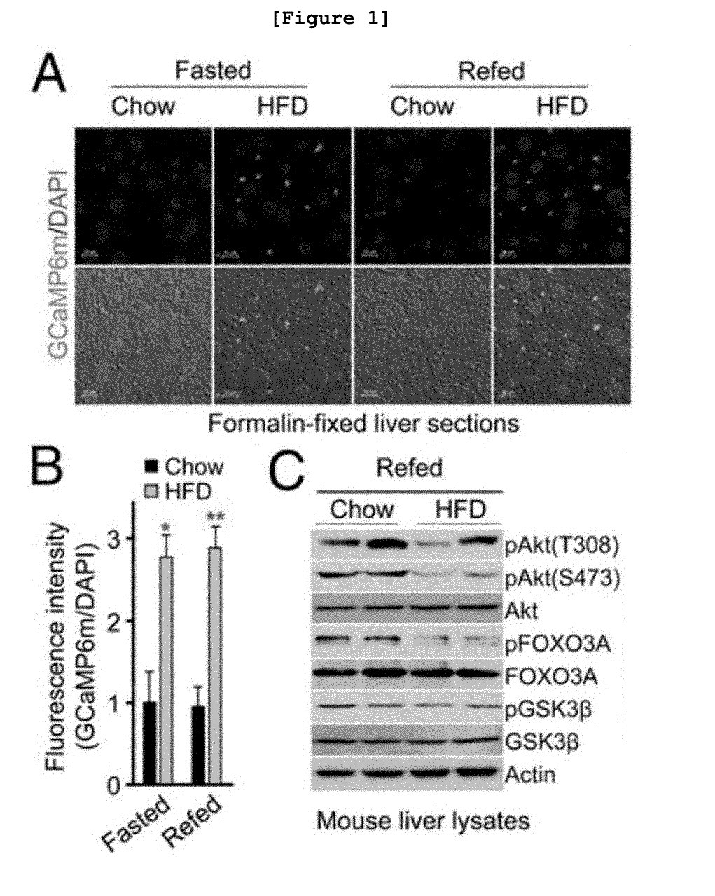

First, male C57BL/6 mice at 8 weeks (Orient Bio, Korea) were raised in an animal facility of Lee Gil Ya Cancer and Diabetes Institute (Gachon University, Korea). Particularly, the mice were raised in a sterile chamber at the temperature of 23±1° C., with 12 hr/12 hr light/dark cycle, and free dietary environment. At this time, the mice were divided into the fasted group wherein the mice were fasted for 16 hours and the refed group wherein the mice were fasted for 16 hours and then fed for 4 hours. Each group was sub-divided into the high fat diet group (HFD) and the normal diet group (chow). The high fat diet group (HFD) mice were fed with diet containing 60% fat, while the normal diet group (chow) mice were fed with diet containing 10% fat. 8 weeks later, the mice were anesthetized and the liver was perfused by using buffer 1 (142 mM NaCl, 6.7 mM KCl, 10 mM HEPES and 2.5 mM EGTA, pH 7.4). Buffer 1 above was replaced with buffer 2 (0.5 mg/Me collagen, 66.7 mM NaCl, 6.7 mM KCl, 10 mM HEPES and 4.8 mM CaCl, pH 7.6), and then the perfused liver was extracted. The extracted liver was washed and degraded by using Percoll cushion gradient. The degraded cells were suspended and cultured in Hepatozyme-SFM (Gibco-BRL, USA) culture medium supplemented with 10% FBS and 1% antibiotics and antifungal agent. The prepared cells were diluted in DMEM at the density of 1×10⁷ cells/Ml, to which 4 μM Fluo-3 AM (Invitrogen, USA) was added. The cells were reacted at 37° C. for 45 minutes and then fixed with 4% paraformaldehyde for 10 minutes. The fixed cells were washed with PBS twice for 5 minutes each time and dried, followed by mounting by using 4,6-diamidino-2-phenylindole. The mounted cells were analyzed using a fluorescence microscope (Zeiss Axioplan 2). The results are shown in FIG. 1A and FIG. 1B.

As shown in FIG. 1A and FIG. 1B, the intracellular calcium level was significantly increased in the liver tissues of the mouse fed with high fat diet 4 times, compared with the mouse fed with normal diet (FIG. 1A and FIG. 1B).

### Example 2: Confirmation of Inhibition of Insulin Signaling in Insulin Resistance Animal Model Induced by Obesity

To investigate the insulin sensitivity in the liver cells of the insulin resistance animal model induced by obesity, the expression patterns of the proteins involved in Akt protein phosphorylation mechanism were confirmed by Western blotting.

First, male C57BL/6 mice at 8 weeks were divided into the fasted group wherein the mice were fasted for 16 hours and the refed group wherein the mice were fasted for 16 hours and then fed for 4 hours. Each group was sub-divided into the high fat diet group (HFD) and the normal diet group (chow). The liver cells were extracted from the raised mice by the same manner and under the same conditions as described in Example 1. The extracted liver cells were lysed in a lysis buffer containing phosphatase (Sigma-Aldrich, USA) and protease inhibitor (Sigma-Aldrich, USA). Proteins extracted from 2 to 3 mg of the liver tissue were homogenized by using a tissue homogenizer (TissuLyser, Qiagen, USA) for 1.5 minutes at a frequency of 1/30 sec. The homogenized lysate was centrifuged at 12,000 rpm for 15 minutes and the supernatant was obtained. The amount of the extracted protein was quantified by using a protein assay kit (Bio-Rad Laboratories, USA), and at this time BSA was used as a standard sample. 3 mg of the protein was mixed with 6× loading buffer, followed by electrophoresis using 10% SDS-PAGE gel. The electrophoresed protein was transferred onto a polyvinylidene fluoride (PVDF) membrane by using a transfer buffer at 4° C. with 120 V for 1.5 hours. The PVDF membrane on which the protein was transferred was pretreated with a blocking buffer prepared by mixing 5% skim milk with TBS buffer (TBS-T, pH 7.6) supplemented with Tween 20 for 30 minutes, and the membrane was washed with TBS-T. The washed membrane was added with the antibody against Akt (Cell Signaling Technology, USA), Akt(T308) (Cell Signaling Technology, USA), Akt(S473) (Cell Signaling Technology, USA), FOXO3A (Cell Signaling Technology, USA), FOXO3A(S253) (Cell Signaling Technology, USA), GSK-3β (Cell Signaling Technology, USA) or GSK-3β(S9) (Cell Signaling Technology, USA) protein as a primary antibody, followed by reaction at 4° C. for 12 hours. At this time, the antibody against actin (Santa Cruz Biotechnology, USA) protein was used for the control group. Upon completion of the reaction, the secondary antibody corresponding to the primary antibody was added thereto, followed by reaction at room temperature for 30 minutes. The PVDF membrane was washed with TBS-T buffer three times. The washed PVDF membrane was added with ECL solution (Thermo Scientific, USA), followed by image observation. Images were photographed by using LAS 4000 imaging system (GE Healthcare, USA), which are shown in FIG. 1C.

As shown in FIG. 1C, the Akt protein phosphorylation and the phosphorylations of the proteins in FOXO3A and GSK-3β signaling pathways located downstream of the Akt protein phosphorylation pathway were reduced in liver cells of the high fat diet group mice (FIG. 1C).

### Example 3: Confirmation of Increased Calcium Levels in Insulin Resistance Induced Cells

The following experiment was performed to investigate whether or not the intracellular calcium level was increased in the fatty liver cell model constructed in vitro by inducing insulin resistance using saturated free fatty acid (FFA).

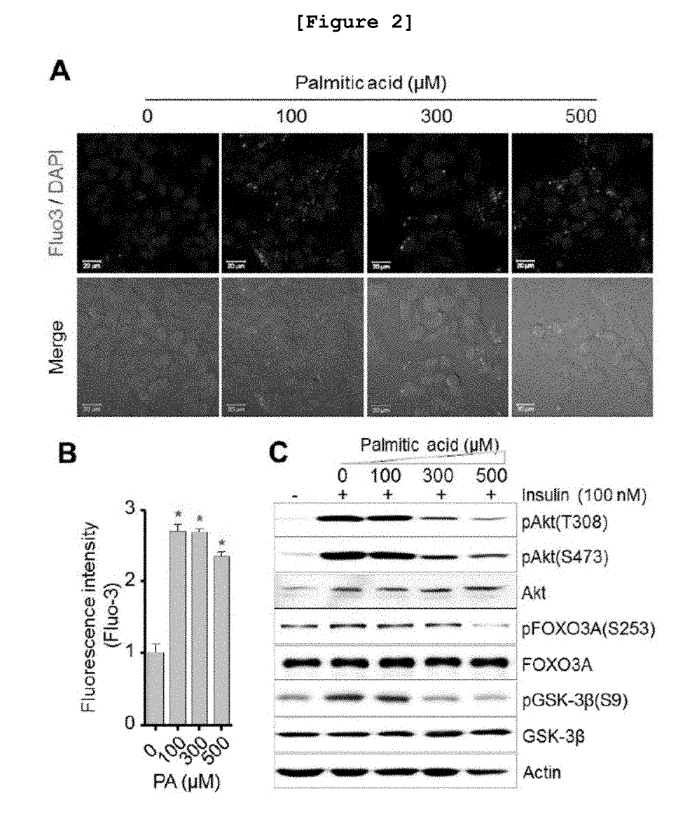

First, human HepG2 cells were cultured in DMEM (Dulbecco's Modified Eagle Medium) supplemented with 10% fetal bovine serum (FBS), 25 mM glucose, 2 mM L-glutamine, 100 U/Ml of penicillin and 100 μg/Ml of streptomycin. At this time, the culture was performed in a 37° C., 5% CO₂ incubator. The cultured cells were distributed in a culture plate at the density of 5.0×10⁵ cells/well, followed by further culture for overnight. Palmitic acid (PA) was added to the plate at the concentration of 0, 100, 300 or 500 μM, followed by reaction for 24 hours. Then, the concentration of calcium was investigated by using Fluo-3 AM according to the same conditions and procedures as described in Example 1, and the results are shown in FIG. 2A and FIG. 2B.

As shown in FIG. 1A and FIG. 2B, the concentration of calcium was increased in the cytoplasm of the HepG2 cells treated with PA (FIG. 2A and FIG. 2B).

### Example 4: Inhibition of Insulin Signaling Mechanism in Insulin Resistance Induced Cells

To investigate the changes in insulin sensitivity when insulin was added to the fatty liver cell model constructed in vitro, the phosphorylations of those proteins that were involved in insulin signaling mechanism were examined. PA was added to the human HepG2 cells prepared in Example 3, followed by reaction for 24 hours. The experiment was performed by the same manner under the same conditions as described in Example 2 except that 100 nM insulin was added thereto 15 minutes before the reaction was completed.

As a result, as shown in FIG. 2C, the Akt protein phosphorylation was significantly inhibited in the PA treated cells, compared with the cells treated with insulin alone, indicating the insulin sensitivity was impaired (FIG. 2C).

### Example 5: Confirmation of Intracellular Calcium Dynamics in Insulin Resistance Induced Cells

Intracellular calcium dynamics was confirmed in the fatty liver cell model constructed in vitro by inducing insulin resistance. At this time, the experiment was performed by the same manner under the same conditions as described in Example 3 except that Fura-2 AM was used instead of Fluo-3 AM.

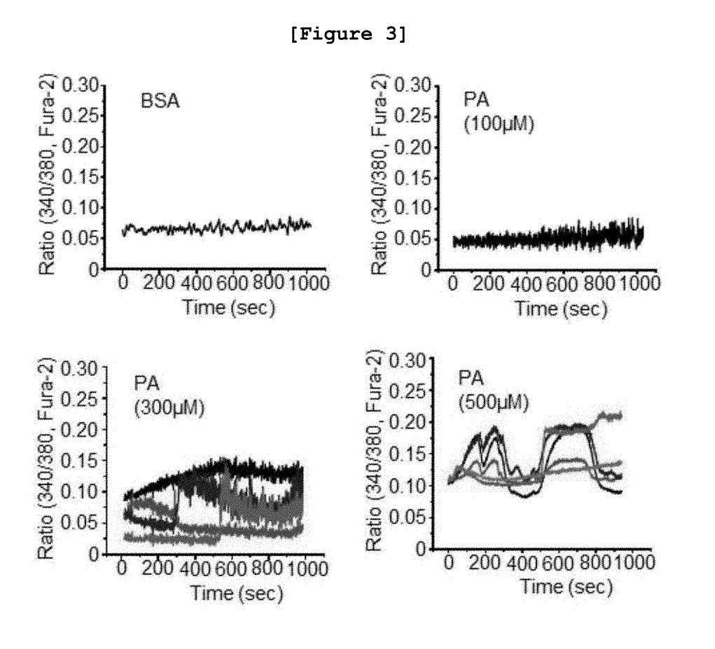

As a result, as shown in FIG. 3, the intracellular calcium concentration was significantly increased by PA (FIG. 3).

### Example 6: Inhibition of Insulin Signaling by Changes of Intracellular Calcium Concentration

### <6-1> Effect of Increase of Calcium

The effect of PMA (phorbol myristate acetate) and ionomycin, the drugs accelerating calcium flux, on insulin sensitivity was investigated. PMA and ionomycin are known to increase intracellular calcium and transmit a strong calcium signal.

For the experiment, human HepG2 cells were treated with PMA at the concentration of 0, 10, 50 or 100 nM instead of PA or with ionomycin at the different concentration of 0, 1, 5, 10 or 20 μM. 100 nM insulin was added thereto 15 minutes before the reaction was completed. The experiment was performed by the same manner under the same conditions as described in Example 2 except that the antibody against pAkt (T308), pAkt (S473), Akt or actin was used as the primary antibody.

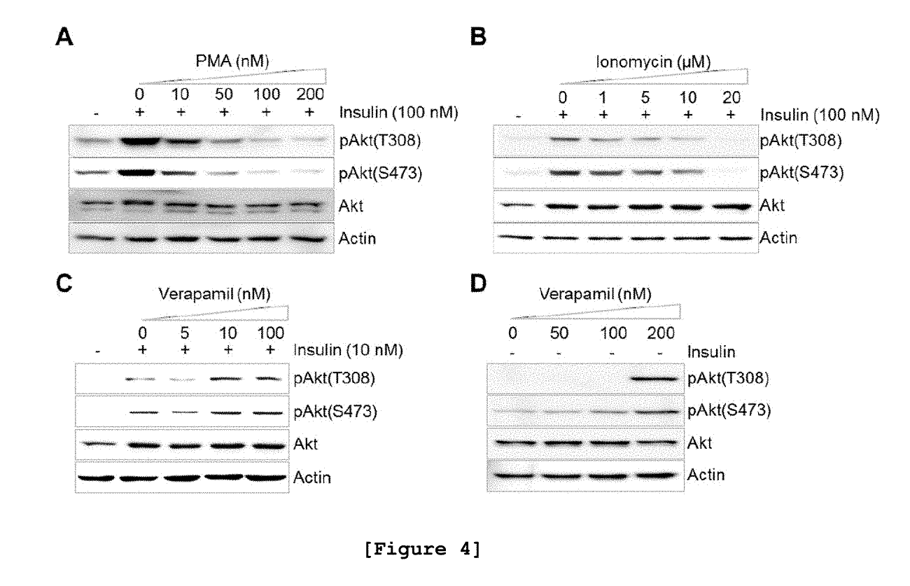

As a result, as shown in FIG. 4A and FIG. 4B, the phosphorylation of Akt protein was significantly inhibited by PMA or ionomycin and at this time the inhibition was dose-dependent (FIG. 4A and FIG. 4B). Therefore, it was confirmed that the insulin sensitivity was impaired by the increase of calcium.

### <6-2> Effect of Decrease of Calcium

To investigate the effect of decrease of calcium on insulin sensitivity, the following experiment was performed using verapamil, the L-type calcium channel blocker. The experiment was performed by the same manner under the same conditions as described in Example <6-1> except that verapamil was added at the concentration of 0, 5, 10 or 100 nM instead of PMA or ionomycin and the effect of insulin addition was investigated.

As a result, as shown in FIG. 4C and FIG. 4D, the phosphorylation of Akt protein was increased by verapamil (FIG. 4C and FIG. 4D), indicating that the insulin sensitivity was increased by the decrease of intracellular calcium.

Therefore, it was confirmed that the changes of intracellular calcium concentration was related to the phosphorylation of Akt protein.

### Example 7: Inhibition of Binding of Akt Protein to PI(3,4)P₂ or PI(3,4,5)P₃ by Calcium

For the activation of Akt protein, Thr308 and Ser473 of Akt protein need to be phosphorylated first and then the PH domain of Akt protein has to bind to PI(3,4,5)P₃, and then the complex must move to the cell membrane.

Thus, the present inventors investigated the effect of calcium on the binding of Akt protein to PI(3,4)P₂ or PI(3,4,5)P₃ by the following method.

### <7-1> Purification of PH Domain of Akt Protein

First, PCR was performed to obtain the PH domain of Akt1 protein (SEQ. ID. NO: 13) by using the primers consisting of the nucleotide sequences represented respectively by SEQ. ID. NO: 3 and SEQ. ID. NO: 4 as listed in Table 1 below. The obtained PCR product was cloned in pET28a vector (Novagen, USA).

TABLE 1

| | | SEQ. |
|---|---|---|
| | | ID. |
| Name | Sequence (5′→3′) | NO: |
| hPKCβ1_C2_PN127 | AAACCATGGGCCACCAGGGGATGAA | SEQ. |
| (NcoI) | ATGTGACACC | ID. |
| | | NO: 1 |
| hPKCβ1_C2_PC296 | AAACTCGAGCAGATCCTCTTCTGAG | SEQ. |
| (XhoI) | ATGAGTTTTTGTTCCTCACTTCCTTC | ID. |
| | TGGTGGCACAGG | NO: 2 |
| hAkt1_PH_PN1 | AAAGAGCTCATGGGCAGCGACGTGG | SEQ. |
| (SacI) | CTAT | ID. |
| | | NO: 3 |
| hAKT1_PH_PC144 | AAAACTAGTTCAGTGGTGGTGGTGG | SEQ. |
| (SpeI) | TGGTGGCGGTGCTTGGGCTTGGCCA | ID. |
| | | NO: 4 |
| hPKCβ1_C2_PN159 | AAAAGATCTATGCGCGGCCGCATCT | SEQ. |
| (BglII) | ACATCCA | ID. |
| | | NO: 5 |
| hPKCβ1_C2_PC289 | AAAGGATCCAGGCACATTGAAGTAC | SEQ. |
| (BanHI) | TCGC | ID. |
| | | NO: 6 |
| hAkt1_PH_PN6 | AAAAGATCTATGATTGTGAAGGAGG | SEQ. |
| (BglII) | GTTGG | ID. |
| | | NO: 7 |
| hAkt1_PH_PN111 | AAAGGATCCCTTGAGGCCGTCAGCC | SEQ. |
| (BamHI) | AC | ID. |
| | | NO: 8 |
| hPKCβ1_C2_PN127 | AAAAGATCTCGCTCATTGTCCTCGT | SEQ. |
| (BglII) | AAGAGA | ID. |
| | | NO: 9 |
| hPKCβ1_C2_PC296 | AAAGCTAGCGGACAAAGATCCCATG | SEQ. |
| (NheI) | AAGT | ID. |
| | | NO: 10 |
| hAKT1_kinase_PN | AAAGCTAGCTTTGAGTACCTGAAGC | SEQ. |
| (NheI) | TGCT | ID. |
| | | NO: 11 |
| hAKT1_kinase_PC | AAAGGTACCTCAGGCCGTGCCGCTG | SEQ. |
| (KpnI) | GCCG | ID. |
| | | NO: 12 |

E. coli BL21 (DE3) cells were transfected with a vector containing the PH domain of Akt protein, and the cells were cultured in 50 Ml of LB medium supplemented with 50 μg/Ml of kanamycin. The culture was performed at 37° C. for 8 hours. Upon completion of the culture, the cells were transferred into a 25° C. agitation incubator. The cells were cultured until OD₆₀₀ reached 0.6 to 1.0, and then 1 mM IPTG (isopropyl-β-D-1-thiogalactopyranoside) was added thereto. 18 hours after the addition of IPTG, centrifugation was performed at 7,000×g at 4° C. for 10 minutes to obtain cells. Sonication equilibrium buffer (150 mM NaCl, 50 mM Tris-HCl, pH 7.6) was added to the obtained cells. The cells added with the sonication equilibrium buffer were lysed by sonication. The PH domain of Akt protein was purified from the cell lysate by gel filtration chromatography using nickel-nitrilotriacetic acid and HiLoad Superdex-200 column (GE Healthcare life science, USA). The purity of the purified protein was examined by SDS-PAGE (sodium dodecyl sulfate polyacrylamide gel electrophoresis).

<7-2> Inhibition of Binding of Akt Protein PH Domain to PI(3,4)P₂ or PI(3,4,5)P₃ by Calcium-1

The effect of calcium on the binding of the PH domain of Akt protein to PI(4,5)P₂ or PI(3,4,5)P₃ was investigated by protein-lipid binding assay.

Particularly, the analysis was performed by using membrane-immobilized lipid strips (PIP-strips, Echelon Biosciences Inc., USA). At this time, the membrane was loaded with LPA (lysophosphatidic acid), LPC (lysophosphatidylcholine), PI (phosphatidylinositol), PE (phosphatidylethanolamine), PC (phosphatidylcholine), S1P (sphingosine 1-phosphate), PA (phosphatidic acid) or PS (phosphatidylserine) at the concentration of 100 pmol, to which calcium was added at the concentration of 0, 0.05 or 0.1 mM. Each membrane was pre-treated with a blocking buffer [fatty acid free 3% BSA (bovine serum albumin), 0.1% (v/v) Tween 20 and TBS (Tris-buffered saline), pH 7.5] at room temperature for 30 minutes. The PH domain of Atk protein purified in Example <7-1> was suspended in a blocking buffer at the concentration of 3.5 mg/Ml. The suspended protein was reacted with the membrane-immobilized lipid strip at room temperature for 30 minutes. The protein bound with lipid was identified by using the PH domain antibody.

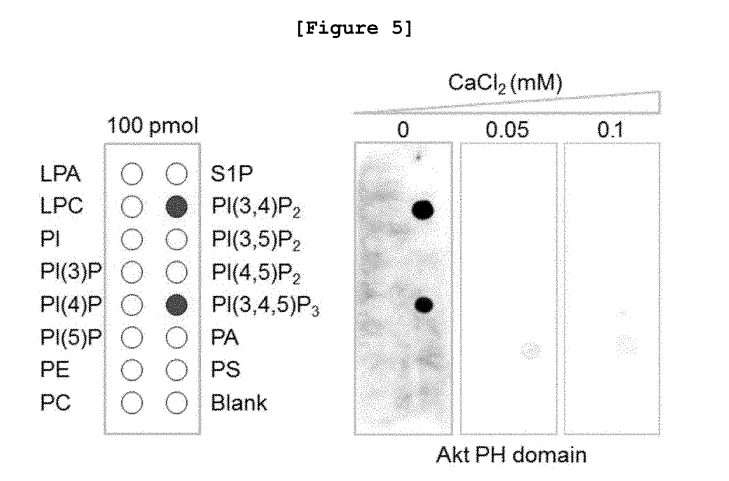

As a result, as shown in FIG. 5, the PH domain of Akt protein was bound to PI(3,4)P₂ or PI(3,4,5)P₃ and the binding was inhibited as calcium concentration was increased (FIG. 5).

<7-3> Inhibition of Binding of Akt Protein PH Domain to PI(3,4)P₂, PI(4,5)P₂ or PI(3,4,5)P₃ by Calcium-2

The effect of calcium on the binding of the PH domain of Akt protein to PI(3,4)P₂, PI(4,5)P₂ or PI(3,4,5)P₃ was investigated by ITC (isothermal titration calorimetry).

Particularly, a protein solution was prepared by suspending a protein in 10 mM HEPES (pH 7.0). Phospholipid related ligand was titrated in the protein solution at 25° C. by using iTC200 micro calorimetry system (MicroCal Inc., USA), and the generated graph was corrected using Origin software (MicroCal Inc., USA). At this time, the phospholipid related ligand was prepared in the form of liposome by mixing POPC (1-palmitoyl-2-oleoyl-sn-glycero-3-phosphocholin) chloroform solution and POPS (1-palmitoyl-2-oleoyl-sn-glycero-3-phosphoserine), PI(3,4)P₂ (1,2-dipalmotoyl-sn-glycero-3-phosphatidylinositol-3,4-triphosphate), PI(4,5)P₂ (1,2-dipalmotoyl-sn-glycero-3-phosphatidylinositol-4,5-trisphosphate) or PI(3,4,5)P₃ (1,2-dipalmotoyl-sn-glycero-3-phosphatidylinositol-3,4,5-trisphosphate) at the molar ratio of 80:20. The organic solvent was eliminated. The organic solvent was removed and a single layer lamellar was produced in HEPES (pH 7.0) using phospholipids.

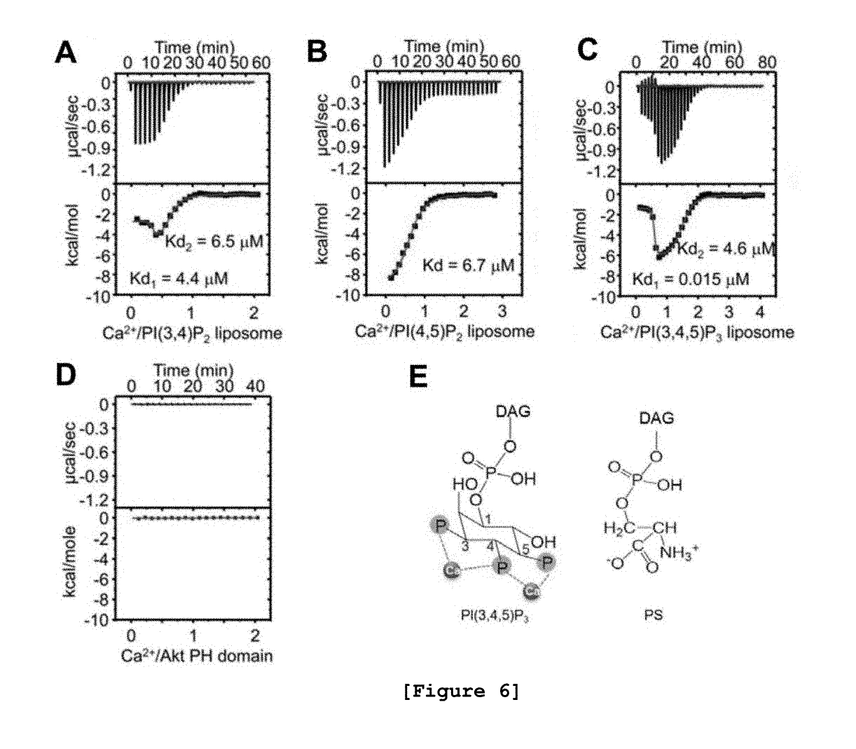

As a result, as shown in FIG. 6A to FIG. 6C, PI(3,4)P₂, PI(4,5)P₂ or PI(3,4,5)P₃ was bound to calcium with a high affinity, unlike POPS (FIG. 6A ˜FIG. 6D). In particular, PI(3,4,5)P₃ had two calcium binding sites (FIG. 6E), so the binding force was further increased.

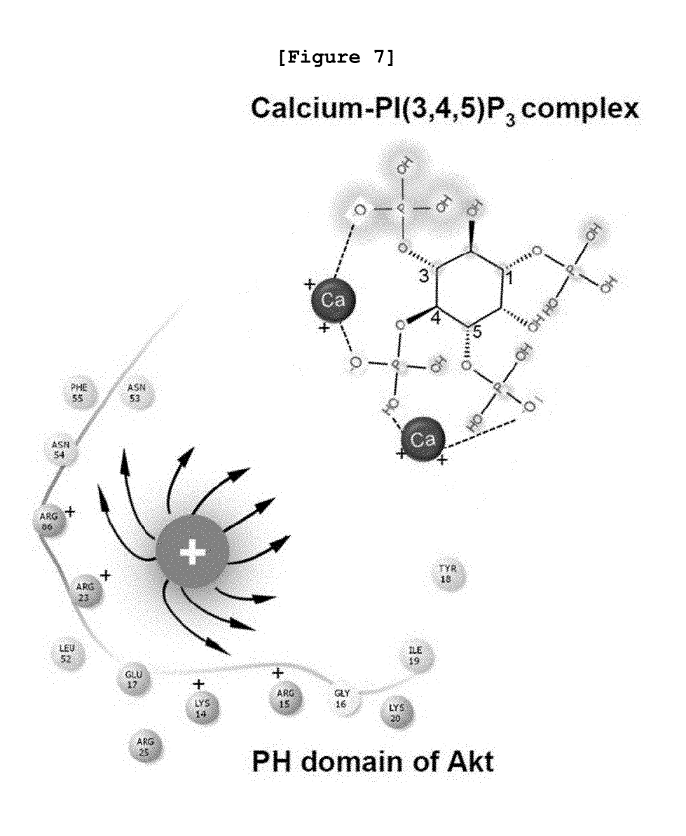

From the above results, it was confirmed that PI(3,4,5)P₃, PI(3,4)P₂ and PI(4,5)P₂ had a strong binding force to calcium and once intracellular calcium was accumulated, the binding force of the Akt protein PH domain thereto was reduced. In a healthy condition, the phosphate group in the carbon ring of PI(3,4,5)P₃ has negative charge and the PH domain has positive charge, so that they are bound together stably. In the obese state, the intracellular calcium concentration is increased, so that calcium is bound to the phosphate group of PI(3,4,5)P₃. Thus, the interaction between PI(3,4,5)P₃ and the PH domain does not occur (FIG. 7).

### Example 8: Confirmation of Binding of PKCβ Protein C2 Domain to PI(4,5)P₂ or PI(3,4,5)P₃

It was investigated whether or not the C2 domain having the characteristic of interacting with calcium like the PH domain of Akt protein was bound to PI(4,5)P₂ or PI(3,4,5)P₃.

The C2 domain above is found in approximately 362 kinds of proteins in human and total 582 C2 domains are known to exist. Particularly, the C2 domain is found in PLC, P13K and PKC proteins. The C2 domain either has a calcium binding site for direct binding to calcium or can bind to PIP calcium independently.

### <8-1> Purification of C2 Domain of PKCβ Protein

The C2 domain of PKCβ protein (SEQ. ID. NO: 14) was purified by the same manner under the same conditions as described in Example <7-1> except that the primers consisting of the nucleotide sequences represented by SEQ. ID. NO: 1 and SEQ. ID. NO: 2 listed in Table 1 were used. In the meantime, D187/193A and D246/248A, the C2 domain mutant forms, were purified as for the control by the same manner.

<8-2> Inhibition of Binding of PKCβ Protein C2 Domain to PI(4,5)P₂ or PI(3,4,5)P₃ by Calcium

To investigate whether or not the binding of the PKCβ protein C2 domain purified in Example <8-1> to PI(4,5)P₂ or PI(3,4,5)P₃ was inhibited by calcium, protein-lipid binding assay and ITC were performed. First, protein-lipid binding assay was performed by the same manner under the same conditions as described in Example <7-2> except that the PKCβ protein C2 domain was used instead of the Akt protein PH domain. At this time, D187/193A and D246/248A mutant proteins of the C2 domain were used as the control group, and the results are shown in FIG. 8.

As shown in FIG. 8, the C2 domain of PKCβ protein was strongly bound to PI(4,5)P₂ and PI(3,4,5)P₃ (FIG. 8).

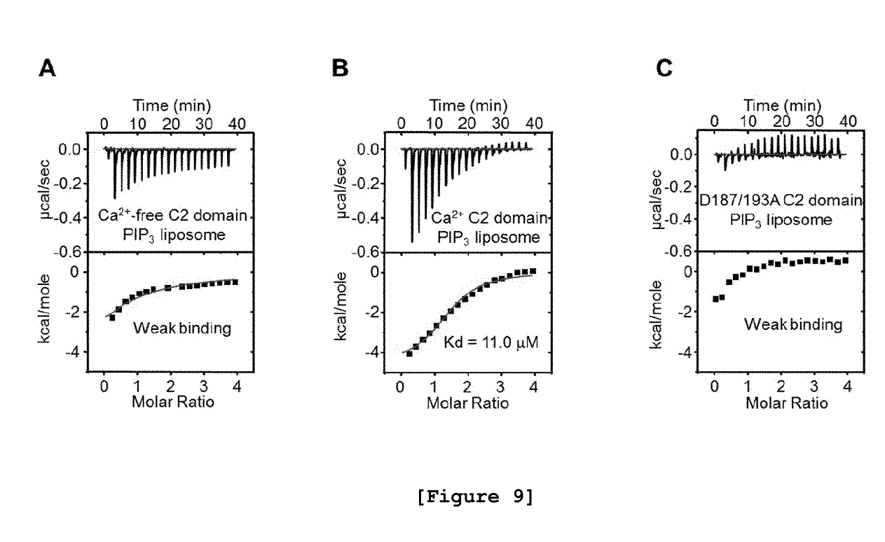

In the meantime, in ITC, the phospholipid related ligand was prepared in the form of liposome by mixing POPC chloroform solution and PI(3,4,5)P₃ at the molar ratio of 80:20. Then, ITC was performed by the same manner under the same conditions as described in Example <7-3> except that the PKCβ protein C2 domain or D187/193A, the C2 domain mutant, was used. The results are shown in FIG. 9.

As shown in FIG. 9B, the PKCβ protein C2 domain was strongly bound to PI(3,4,5)P₃ (FIG. 9B). However, the C2 domain mutant form D187/193A which had a mutation on the calcium binding site of the C2 domain was barely bound to PI(3,4,5)P₃ (FIG. 9C). Therefore, it was confirmed that D187 residue and D193 residue in the C2 domain were the ones that directly interacted with calcium.

### Example 9: Migration of PH or C2 Domain According to Calcium Concentration

From the above results, it was confirmed that the PH domain of Akt protein or the C2 domain of PKCβ protein was bound to the cell membrane. Thus, the present inventors performed immunostaining to investigate the effect of calcium concentration change on the binding of such domains above to the cell membrane.

First, the PH domain of Akt1 protein or the C2 domain of PKCβ protein was obtained by PCR using the primers consisting of the nucleotide sequences represented by SEQ. ID. NO: 5˜SEQ. ID. NO: 8 listed in Table 1 above. The obtained PCR product was cloned in pmCherry-C1 vector (Takara Clontech, Japan) by the conventional cloning method. CHO cells were transfected with the prepared vector by using lipofectamine according to the manufacturer's protocol. The transfected cells were selected by using blastidicin. Then, mCherry positive cells were confirmed by flow cytometry.

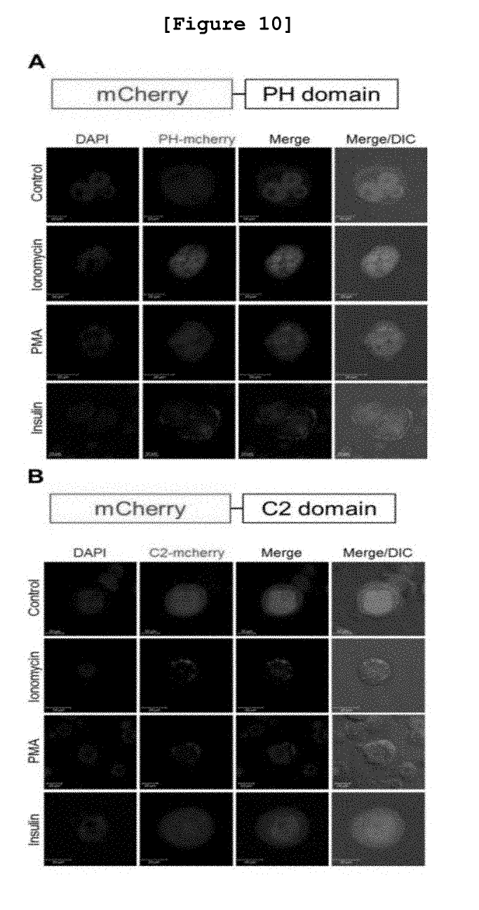

PMA at the concentration of 100 nM, ionomycin at the concentration of 10 μM or insulin at the concentration of 100 nM was added to the cells expressing the PH domain of Akt1 protein or the C2 domain of PKCβ protein and the location of the expressed protein was confirmed by fluorescence microscopy. The results are shown in FIG. 10.

As shown in FIG. 10A, the PH domain migrated to the cell membrane by the addition of insulin. When the intracellular calcium concentration was increased, the migration to the cell membrane was not observed (FIG. 10A). However as shown in FIG. 10B, the C2 domain did not migrate to the cell membrane even when insulin was added, but migrated as the intracellular calcium concentration was increased (FIG. 10B).

From the above results, it was confirmed that when the calcium concentration was increased, the binding of the PH domain of Akt protein to PI(3,4,5)P₃ was inhibited but the binding of the C2 domain to PI(3,4,5)P₃ was not affected even under the condition of increased calcium because the C2 domain was interacted with the up-regulated calcium as well.

### Example 10: Effect of PKCβ Protein C2 Domain and Akt Protein Fusion Protein on Akt Activation

A fusion protein was prepared by using the C2 domain of PKCβ protein instead of the PH domain of Akt protein. It was investigated that the Akt activation was dependent on PI3K under the condition of increased calcium concentration induced by the fusion protein.

First, the C2 domain of PKCβ protein and Akt protein (SEQ. ID. NO: 15) were obtained by PCR using the primers consisting of the nucleotide sequences represented by SEQ. ID. NO: 9˜SEQ. ID. NO: 12 listed in Table 1 above. The obtained PCR product was cloned in pEGFP-C2 vector by the conventional cloning method, resulting in the construction of a vector expressing the C2 domain/Akt protein fusion protein (SEQ. ID. NO: 17) (C2-Akt fusion protein). At this time, as the control group, a vector expressing the wild type Akt protein containing the PH domain was prepared.

HepG2 cells were transfected with the vector expressing the C2-Akt fusion protein constructed above along with the vector expressing P13K protein by using lipofectamine according to the manufacturer's protocol. The transfected cells were treated with PMA at the concentration of 0, 10, 50 or 100 nM ionomycin at the concentration of 20 μM, followed by reaction for 1 hour. Western blot analysis was performed with the cells above. The experiment was performed by the same manner under the same conditions as described in Example 2 except that the antibody against Akt (Cell Signaling Technology, USA), Akt(T308) (Cell Signaling Technology, USA), Akt(S473) (Cell Signaling Technology, USA), FOXO3A (Cell Signaling Technology, USA), FOXO3A(S253) (Cell Signaling Technology, USA), GSK-30 (Cell Signaling Technology, USA), GSK-3β(S9) (Cell Signaling Technology, USA) or PI3K (Cell Signaling Technology, USA) protein was used as the primary antibody.

As a result, as shown in FIG. 11 and FIG. 12, Akt protein was activated by P13K in the cells transfected with the wild type Akt protein and the activation was inhibited as the calcium concentration increased. However, the Akt protein activation was not inhibited but increased by the increase of calcium concentration in the cells transfected with the C2-Akt fusion protein (FIG. 11 and FIG. 12).

### Example 11: Confirmation of Signal Transduction Mechanism by PKCβ Protein C2 Domain and Akt Protein Fusion Protein

The following experiment was performed to investigate which of PI(4,5)P₂ and PI(3,4,5)P₃P1 was more important for the activation of Akt protein by the C2-Akt fusion protein.

Particularly, the experiment was performed by the same manner under the same conditions as described in Example 2 except that the vector expressing the C2-Akt fusion protein and the vector expressing PI3K protein were co-treated with PTEN or SHIP2 and the antibody against Akt (Cell Signaling Technology, USA), Akt(T308) (Cell Signaling Technology, USA), Akt(S473) (Cell Signaling Technology, USA), PI3K (Cell Signaling Technology, USA), PTEN (Cell Signaling Technology, USA) or SHIP2 (Upstate Biotechnology, USA) protein was used as the primary antibody.

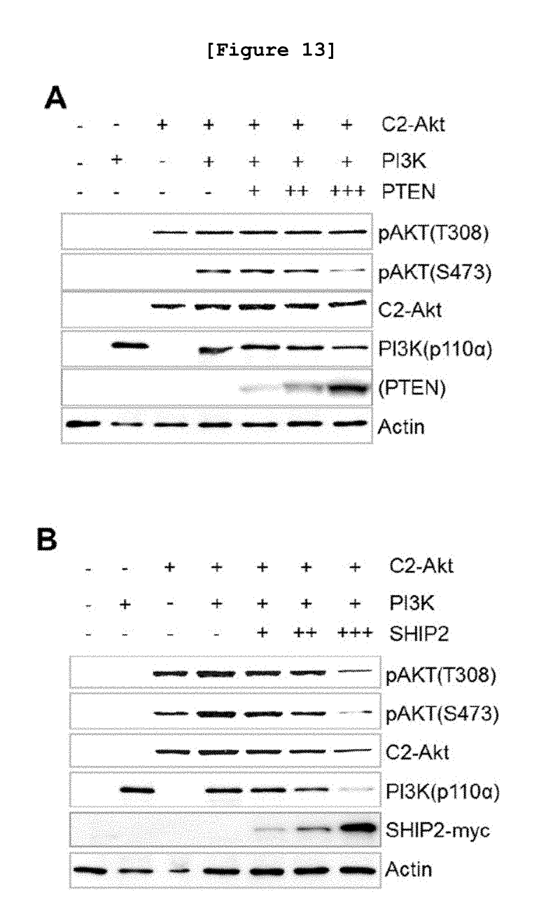

As a result, as shown in FIG. 13, the Akt protein activity was reduced by the increase of intracellular PI(4,5)P₂ induced by PTEN or SHIP2 (FIG. 13). So, it was confirmed that PI(3,4,5)P₃ was more important than PI(4,5)P₂ in changing the Akt protein activation by the C2-Akt fusion protein.

### Example 12: Confirmation of C2 Domain/Calcium Binding Site in PKCβ Protein C2 Domain and Akt Protein Fusion Protein

From the above results, it was confirmed that the C2-Akt fusion protein was able to activate Akt by binding to PIP conjugated calcium even under the condition of high calcium concentration. Therefore, the following experiment was performed to investigate which residue among the calcium binding residues existing in the C2 domain played the most important role.

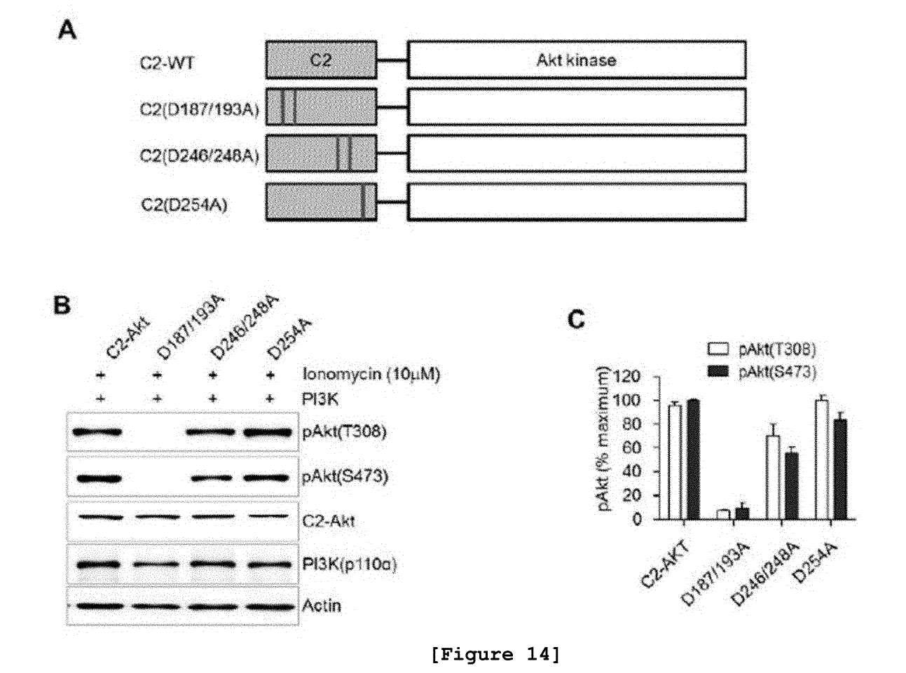

First, a mutant form of the C2-Akt fusion protein was constructed by mutating a residue known as a calcium binding site in the C2-Akt fusion protein C2 domain. Particularly, D187/193A, D246/248A or D254T mutant from of the C2 domain was constructed (FIG. 14A). HepG2 cells were transfected with the vector expressing P13K protein together with the C2-Akt fusion protein mutant form by using lipofectamine according to the manufacturer's protocol. The transfected cells were treated with ionomycin at the concentration of 10 μM, followed by reaction for 1 hour. Western blot analysis was performed with the cells above. The experiment was performed by the same manner under the same conditions as described in Example 2 except that the antibody against Akt (Cell Signaling Technology, USA), Akt(T308) (Cell Signaling Technology, USA), Akt(S473) (Cell Signaling Technology, USA) or PI3K (Cell Signaling Technology, USA) protein was used as the primary antibody.

As a result, as shown in FIG. 14B and FIG. 14C, the D246/248A or D254 mutant fusion protein was still able to activate Akt protein in the presence of P13K, but the D187/193A mutant did not activate Akt protein. From the above results, it was confirmed that the D187 and D193 residues of the C2 domain were important for the C2-Akt fusion protein to activate Akt protein under the condition of high calcium concentration.

### Example 13: Treatment Effect of PKCβ Protein C2 Domain and Akt Protein Fusion Protein on Obesity Induced Insulin Resistance

<13-1> Confirmation of Infection with Adenovirus Expressing PKCβ Protein C2 Domain and Akt Protein Fusion Protein and Weight Changes of Animal Model Infected with Adenovirus Thereof

The treatment effect of the C2-Akt fusion protein on insulin resistance in the insulin resistance animal model induced by high fat diet mediated obesity was investigated as follows.

First, male C57BL/6 mice at 8 weeks were fed with HFD diet (60% high fat diet) for 8 weeks to construct an obesity-induced insulin resistance animal model.

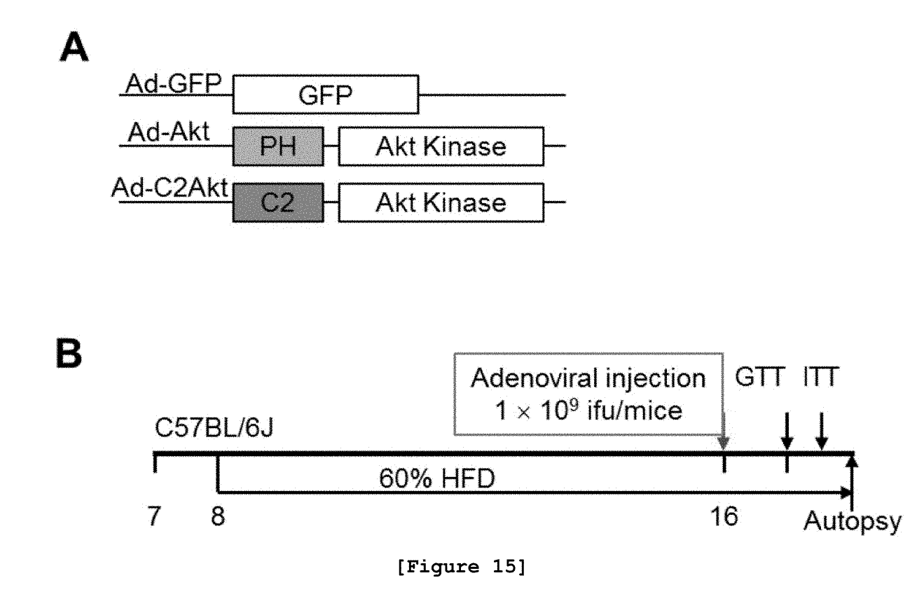

In the meantime, Ad-Akt and Ad-C2Akt expression vectors were prepared by cloning the wild type Akt protein or the C2-Akt fusion protein into adenovirus vector (FIG. 15A). At this time, Ad-GFP, the expression vector expressing GFP, was constructed as a control. AD-293 cells (Invitrogen, USA) were infected with the constructed vector above by the conventional method well known to those in the art, and the virus was amplified. The amplified viruses were purified by using ViraBind-Adenovirus Purification Kit (Cell Biolabs Inc., USA) according to the manufacturer's protocol. The purified virus was titrated. The constructed mouse model was administered with the virus at the dose of 1×10⁹ PFU through intravenous injection. The mouse injected with the virus was observed for 2 weeks, followed by autopsy (FIG. 15B).

For 2 weeks after the virus injection, the weight change and the feed intake of the mouse were observed and the results are shown in FIG. 16. The fat amount and the changes of dry weight were observed and the results are shown in FIG. 17. As shown in FIG. 16, the body weight of the mouse infected with the adenovirus vector expressing the C2-Akt fusion protein was reduced (FIG. 16A), but the feed intake was not much different from that of the control mouse (FIG. 16B). As shown in FIG. 17, the fat amount and the dry weight of the mouse infected with the adenovirus vector expressing the C2-Akt fusion protein were reduced, compared with the control (FIG. 17).

Therefore, it was confirmed that obesity in the insulin resistance animal model induced by high fat diet mediated obesity was improved by the C2-Akt fusion protein.

### <13-2> Confirmation of Glucose Tolerance and Insulin Resistance According to PKCβ Protein C2 Domain and Akt Protein Fusion Protein

Glucose tolerance and insulin resistance in the mouse infected with the adenovirus vector expressing the C2-Akt fusion protein prepared in Example <13-1> were examined by the following method.

Particularly, the adenovirus vector was injected in the mouse and then the mouse was fasted for 16 hours. The fasted mouse was intraperitoneally injected with glucose (Fisher Scientific, USA) at the concentration of 1 g per 1 kg of the mouse weight (Fisher Scientific, USA). Blood was collected from the tail vein of the mouse model injected with glucose, and the glucose level in the blood sample was measured using a glucose monitor. The results are shown in FIG. 18A and FIG. 18B.

In the meantime, the adenovirus vector was injected into the mouse model and the mouse was fasted for 4 hours. The fasted mouse was intraperitoneally injected with insulin at the concentration of 0.65 unit per 1 kg of the mouse weight. The blood sample was allowed to settle on ice for 1 to 2 hours to coagulate. The coagulated blood was centrifuged at 8,000 rpm at 4° C. for 10 minutes, and only serum was obtained. The insulin level in the obtained serum was measured by using an ELISA kit (Millipore, USA) according to the manufacturer's protocol. The results are shown in FIG. 18C and FIG. 18D.

As shown in FIG. 18A and FIG. 18B, the glucose level in the mouse infected with the adenovirus vector expressing the C2-Akt fusion protein was lower than that of the control (FIG. 18A and FIG. 18B) but the insulin level was not significantly different (FIG. 18C and FIG. 18D).

### <13-3> Confirmation of HOMA-IR Changes According to PKCβ Protein C2 Domain and Akt Protein Fusion Protein

To examine the glucose tolerance and insulin resistance in Example <13-2>, blood samples were taken from the fasting animal model before the injection of glucose or insulin to determine fasting glucose or insulin levels. HOMA-IR (homeostatic model assessment of insulin resistance) values were calculated from the glucose and insulin levels obtained above. The experiment was performed by the same manner under the same conditions as described in Example <13-2> except that the blood sample was obtained from the fasting mouse. HOMA-IR values were calculated by the mathematical formula 1 below with the measured glucose and insulin levels.

HOMA-IR=fasting blood glucose (mg/dl)×fasting insulin (μU/Ml)/22.5 [Mathematical Formula 1]

As a result, as shown in FIG. 19, it was confirmed that the fasting glucose and insulin levels were reduced by the C2-Akt fusion protein. It was also confirmed from the HOMA-IR results that the insulin resistance was improved by the C2-Akt fusion protein (FIG. 19).

### <13-4> Confirmation of Changes in Blood Component According to PKCβ Protein C2 Domain and Akt Protein Fusion Protein

Blood samples were collected from the mouse injected with the adenovirus vector expressing the C2-Akt fusion protein in Example <13-1>, from which the levels of ALT (alanine aminotransferase), total cholesterol and LDL (low density lipoprotein) were measured.

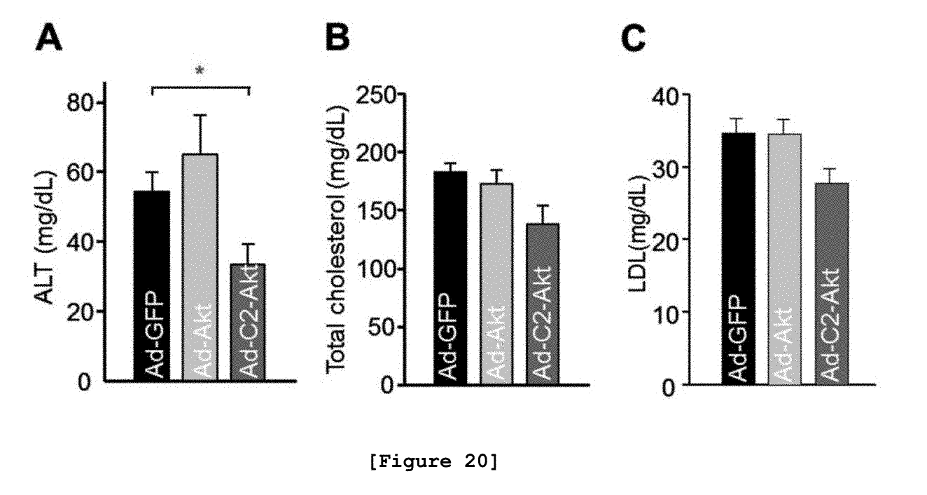

Particularly, the obtained blood sample was centrifuged at 3,000 rpm at 4° C. for 15 minutes, and only serum was obtained. The levels of ALT, total cholesterol and LDL in the obtained serum were measured by using an AU480 chemical analyzer (Beckman Coulter, USA) and the results are shown in FIG. 20.

As shown in FIG. 20, the levels of ALT, total cholesterol and LDL were all reduced in the mouse infected with the adenovirus vector expressing the C2-Akt fusion protein (FIG. 20). Therefore, it was confirmed that the C2-Akt fusion protein was able to improve the liver function of the insulin resistance animal model induced by high fat diet mediated obesity.

### <13-5> Confirmation of Reduction of Liver Lipid Droplets According to PKCβ Protein C2 Domain and Akt Protein Fusion Protein

Liver tissues were extracted from the mouse infected with the adenovirus vector expressing the C2-Akt fusion protein prepared in Example <13-1>, followed by H&E staining to confirm the reduction of liver lipid droplets.

Particularly, the liver was extracted from the mouse (FIG. 21A), which was then fixed in 10% (v/v) neutral formalin buffer. One week later, the liver was sliced to obtain the sections with a thickness of 5 m, which were embedded in paraffin. The embedded tissue was loaded in xylene to eliminate wax, followed by rehydration with alcohol solution. The rehydrated liver tissue sections were mounted on the slide glass, followed by H&E (hematoxylin & eosin) staining. Then, lipid droplets were observed under microscope and the results are shown in FIG. 21B.

As shown in FIG. 21B, the lipid droplets were significantly reduced in the mouse infected with the adenovirus vector expressing the C2-Akt fusion protein, unlike the control mouse (FIG. 21B). From the above results, it was confirmed that the C2-Akt fusion protein had the effect of improving the fatty liver induced by high fat diet by inducing the normal Akt signaling mechanism.

### <13-6> Insulin Signaling Mechanism in Liver According to PKCβ Protein C2 Domain and Akt Protein Fusion Protein

The Insulin signaling mechanism was investigated by the same manner under the same conditions as described in Example 2 except that the liver tissues obtained from the mouse infected with the adenovirus vector expressing the C2-Akt fusion protein prepared in Example <13-1> were used.

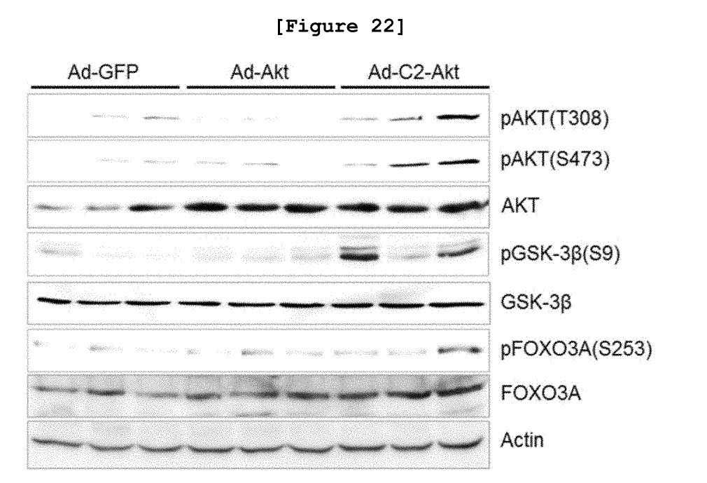

As a result, as shown in FIG. 22, the phosphorylation levels of Akt, GSK3β and FOXO3A were increased in the mouse infected with the adenovirus vector expressing the C2-Akt fusion protein. Therefore, it was confirmed that the C2-Akt fusion protein activated Akt and thus moved the C2-Akt fusion protein to the membrane even in the high fat diet mouse model showing a higher intracellular calcium concentration.

### <13-7> Confirmation of Expression Patterns of Fatty Liver Related Genes According to PKCβ Protein C2 Domain and Akt Protein Fusion Protein

Real time PCR was performed with the liver tissues obtained from the mouse infected with the adenovirus vector expressing the C2-Akt fusion protein obtained in Example <13-1> in order to investigate the expression patterns of the genes involved in glucose synthesis, fat synthesis or fat absorption.

Particularly, 1 Ml of trizol (Invitrogen, USA) was added to 13 mg of the liver tissues obtained in Example <13-1>. The liver tissues treated with trizol were lysed by using a tissue homogenizer, to which 200 μl of chloroform was added. The lysate was centrifuged at 12,000 rpm for 20 minutes to obtain a supernatant. 500 μl of isopropanol was added to the obtained supernatant, followed by vortexing. The reaction mixture stood at room temperature for 10 minutes, and then proceeded to centrifugation at 12,000 rpm for 15 minutes to obtain a pellet. The obtained pellet was washed with ethanol, and then dried at room temperature. The pellet was suspended in distilled water treated with 40 μl of DEPC (diethylpyrocarbonate), from which RNA was isolated. The isolated RNA was quantified by using a nano-drop UV-bis spectrophotometer (Thermo Scientific, USA) and then stored at −80° C. until use.

From the isolated RNA, cDNA was synthesized using a cDNA synthesis kit (Takara Bio, Japan) according to the manufacturer's protocol. Particularly, a proper amount of DNaseI was added to 4 μg of RNA, followed by reaction at 37° C. for 30 minutes and another further reaction at 75° C. for 10 minutes. Upon completion of the reaction, 1 μl of random hexamer primer, 1 μl of dNTP mixture and 3 μl of sterilized distilled water were added to 5 μl of the reaction solution above, followed by further reaction at 65° C. for 5 minutes. 4 μl of 5× PrimeScript buffer, 0.5 μl of RNase inhibitor, 1 μl of RNase and 4.5 μl of sterilized distilled water were added to the reaction solution above, followed by reaction at 30° C. for 10 minutes, at 42° C. for 40 minutes and at 72° C. for 10 minutes. Upon completion of the reaction, 80 μl of sterilized distilled water was added thereto and the synthesized cDNA was stored at −20° C. until use. Particularly, qRT-PCR (quantitative real-time PCR) was performed by using CFX384 real-time PCR detection system (Bio-Rad, USA) with the cycle of reaction at 95° C. for 10 minutes, at 95° C. for 15 minutes (40 times) and 60° C. for 1 minute. At this time, the primers known to be capable of detecting G6PC, PEPCK, SREBPIC, FAS, AccI, Acc2, ChREBP, Mttp, CD36, Ldlr and CPT1 genes were used as the primers for detecting each gene. The relative expression levels of the gene products obtained by qRT-PCR were normalized on the basis of the expression level of cyclophilin gene and calculated by ΔΔCt method.

As a result, as shown in FIG. 23A to FIG. 23C, the expressions of G6PC, PEPCK, SREBP1C, FAS, Acc1, Acc2, ChREBP, Mttp, CD36, Ldlr and CPT1, the genes involved in glucose synthesis, fat synthesis and fat absorption, were reduced in the mouse infected with the adenovirus vector expressing the C2-Akt fusion protein (FIG. 23A˜FIG. 23C).

### <13-8> Confirmation of Expression Patterns of Fatty Liver Related Proteins According to PKCβ Protein C2 Domain and Akt Protein Fusion Protein

Western blotting was performed to investigate whether or not the inhibition of those genes involved in glucose synthesis, fat synthesis or fat absorption in the liver of the mouse treated with high fat diet induced by the C2-Akt fusion protein affected on the protein expression.

The experiment was performed by the same manner under the same conditions as described in Example 2 except that the liver tissues obtained from the mouse infected with the adenovirus vector expressing the C2-Akt fusion protein were used and the antibodies against pSREBP-1 (Santa Cruz Biotechnology, USA), nSREBP-1 (Santa Cruz Biotechnology, USA), FAS (Santa Cruz Biotechnology, USA), ACC1 (Santa Cruz Biotechnology, USA) and ACC2 (Santa Cruz Biotechnology, USA) proteins were used as the primary antibodies.

As a result, as shown in FIG. 23D, the expressions of ACC1 and ACC2 proteins were reduced in the mouse infected with the adenovirus vector expressing the C2-Akt fusion protein (FIG. 23D).

### Example 14: Confirmation of Gene Expression Changes According to PKCβ Protein C2 Domain and Akt Protein Fusion Protein

The expression changes of RNA expressed in the liver of the mouse infected with the adenovirus vector expressing the C2-Akt fusion protein prepared in Example <13-1> were investigated by RNA sequencing. As a result, as shown in FIG. 24A, 5 genes that were up-regulated by the C2-Akt fusion protein and 7 genes that were down-regulated by the fusion protein were confirmed.

The following experiment was performed to investigate the gene expression patterns by the same manner under the same conditions as described in Example <13-5> except that the liver tissues of the mouse and the known primers known to be capable of detecting LCN2, Selenbp2, FABP5, Orm2, FGFR1, Acot3, RGS16, Lpin1, Vnn1, Klf10, Cidea, Cideb or CideC genes were used.

As a result, as shown in FIG. 24B to FIG. 24D, the expressions of the genes playing a role in improving fatty liver such as LCN2, Selenbp2, FABP5, Orm2 and Fgfr1 were increased by the C2-Akt fusion protein, while the expressions of the genes known to accelerate fatty liver such as Acot3, Rgs16, Lpin1, Vnn1 and Klf10 were decreased (FIG. 24B and FIG. 24C). In the meantime, the expressions of the genes involved in lipid droplet production and fat accumulation such as Cidea (cell death inducing DNA fragmentation factor-alpha-like effector protein a) and Cidec were also decreased (FIG. 24D).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

1. A fusion protein consisting of a C2 domain and an Akt protein fragment consisting of the amino acid residues ranging from the 111^{th} to 480^{th} amino acid from the N-terminus of Akt protein.

2. The fusion protein according to claim 1, wherein the C2 domain is originated from PI3K (phosphatidylinositol 3-kinase), PKC (protein kinase C), ABR (active breakpoint cluster region-related), BAIAP3 (BAI1-associated protein 3), BCR (breakpoint cluster region), C2CD2 (C2 calcium dependent domain containing 2), C2CD3 (C2 calcium dependent domain containing 3), CADPS (calcium-dependent secretion activator 1), CADPS2 (calcium-dependent secretion activator 2), CAPN5 (calpain-5), CAPN6 (calpain-6), CC2D1A (coiled-coil and C2 domain-containing protein 1A), CC2D1B (coiled-coil and C2 domain-containing protein 1B), CPNE1 (copine-1), CPNE2 (copine-2), CPNE3 (copine-3), CPNE4 (copine-4), CPNE5 (copine-5), CPNE6 (copine-6), CPNE7 (copine-7), CPNE8 (copine-8), CPNE9 (copine-9), DAB2IP (disabled homolog 2-interacting protein), DOC2A (double C2-like domain-containing protein alpha), DOC2B (double C2-like domain-containing protein beta), DYSF (dysferlin), ESYT1 (extended synaptotagmin-1), ESYT3 (extended synaptotagmin-3), FAM62B (extended synaptotagmin-2), FER1L3 (myoferlin), FER1L5 (fer-1 like family member 5), HECW1 (C2 and WW domain containing E3 ubiquitin protein ligase 1), HECW2 (C2 and WW domain containing E3 ubiquitin protein ligase 1), ITCH (itchy E3 ubiquitin protein ligase), ITSN1 (intersectin-1), ITSN2 (intersectin-2), MCTP1 (multiple C2 and transmembrane domain containing 1), MCTP2 (multiple C2 and transmembrane domain containing 2), MTAC2D1 (tandem C2 domains nuclear protein), NEDD4 (neural precursor cell expressed developmentally down-regulated protein 4), NEDD4L (neural precursor cell expressed developmentally down-regulated gene 4-like), OTOF (otoferlin), PCLO (protein piccolo), PIK3C2A (phosphatidylinositol-4-phosphate 3-kinase C2 domain-containing alpha), PIK3C2B (phosphatidylinositol-4-phosphate 3-kinase C2 domain-containing beta), PIK3C2G (phosphatidylinositol-4-phosphate 3-kinase C2 domain-containing gamma), PLA2G4A (cytosolic phospholipase A2), PLA2G4B (cytosolic phospholipase A2 beta), PLA2G4D (cytosolic phospholipase A2 delta), PLA2G4E (cytosolic phospholipase A2 epsilon), PLA2G4F (cytosolic phospholipase A2 zeta), PLCB1 (1-phosphatidylinositol-4,5-bisphosphate phosphodiesterase beta-1), PLCB2 (1-phosphatidylinositol-4,5-bisphosphate phosphodiesterase beta-2), PLCB3 (1-phosphatidylinositol-4,5-bisphosphate phosphodiesterase beta-3), PLCB4 (1-phosphatidylinositol-4,5-bisphosphate phosphodiesterase beta-4), PLCD1 (phospholipase C delta 1), PLCD3 (phospholipase C delta 3), PLCD4 (phospholipase C delta 4), PLCE1 (phospholipase C epsilon 1), PLCG1 (phospholipase C gamma 1), PLCG2 (phospholipase C gamma 2), PLCH1 (phospholipase C eta 1), PLCH2 (phospholipase C eta 2), PLCL1 (phospholipase C like 1), PLCL2 (phospholipase C like 2), PLCZ1 (phospholipase C zeta 1), PRF1 (perforin-1), PRKCA (protein kinase C alpha), PRKCB1 (protein kinase C beta type), PRKCE (protein kinase C epsilon), PRKCG (protein kinase C gamma), PRKCH (protein kinase C eta), RAB11FIP1 (Rab11 family-interacting protein 1), RAB11FIP2 (Rab11 family-interacting protein 2), RAB11FIP5 (Rab11 family-interacting protein 5), RASA1 (RAS p21 protein activator 1), RASA2 (RAS p21 protein activator 2), RASA3 (RAS p21 protein activator 3), RASA4 (RAS p21 protein activator 4), RASAL1 (RAS protein activator like 1), RASAL2 (RAS protein activator like 2), RGS3 (regulator of G-protein signaling 3), RIMS1 (regulating synaptic membrane exocytosis protein 1), RIMS2 (regulating synaptic membrane exocytosis protein 2), RIMS3 (regulating synaptic membrane exocytosis protein 3), RIMS4 (regulating synaptic membrane exocytosis protein 4), RPGRIP1 (X-linked retinitis pigmentosa GTPase regulator-interacting protein 1), RPH3A (rabphilin-3A), SMURF1 (E3 ubiquitin-protein ligase SMURF1), SMURF2 (E3 ubiquitin-protein ligase SMURF2), SYNGAP1 (synaptic Ras GTPase-activating protein 1), SYT1 (synaptotagmin-1), SYT10 (synaptotagmin-10), SYT11 (synaptotagmin-11), SYT12 (synaptotagmin-12), SYT13 (synaptotagmin-13), SYT14 (synaptotagmin-14), SYT15 (synaptotagmin-15), SYT16 (synaptotagmin-16), SYT17 (synaptotagmin-17), SYT2 (synaptotagmin-2), SYT3 (synaptotagmin-3), SYT4 (synaptotagmin-4), SYT5 (synaptotagmin-5), SYT6 (synaptotagmin-6), SYT7 (synaptotagmin-7), SYT8 (synaptotagmin-8), SYT9 (synaptotagmin-9), SYTL1 (synaptotagmin like 1), SYTL2 (synaptotagmin like 2), SYTL3 (synaptotagmin like 3), SYTL4 (synaptotagmin like 4), SYTL5 (synaptotagmin like 5), TOLLIP (toll interacting protein), UNC13A (Unc-13 homolog A), UNC13B (Unc-13 homolog B), UNC13C (Unc-13 homolog C), UNC13D (Unc-13 homolog D), WWC2 (WW and C2 domain containing 2), WWP1 (WW domain containing E3 ubiquitin protein ligase 1), WWP2 (WW domain containing E3 ubiquitin protein ligase 2) or PTEN (phosphatase and tensin homolog) protein.

3. The fusion protein according to claim 1, wherein the C2 domain is a polypeptide consisting of the amino acid sequence represented by SEQ. ID. NO: 14.

4. The fusion protein according to claim 1, wherein the Akt protein is a polypeptide consisting of the amino acid sequence represented by SEQ. ID. NO: 16.

5. The fusion protein according to claim 1, wherein the fusion protein is a polypeptide consisting of the amino acid sequence represented by SEQ. ID. NO: 17.

6. A polynucleotide encoding the fusion protein of claim 1.

7. (canceled)

8. (canceled)

9. A method for preventing or treating metabolic disease, comprising administering a pharmaceutically effective amount of a fusion protein consisting of a C2 domain and an Akt protein fragment consisting of the amino acid residues ranging from the 111^{th} to 480^{th} amino acid from the N-terminus of Akt protein a polynucleotide encoding the fusion protein, an expression vector comprising the polynucleotide or a host cell transfected with the expression vector.

10. The method according to claim 9, wherein the metabolic disease is obesity, insulin resistance, diabetes or fatty liver.

11. (canceled)

12. A method for screening a candidate substance for treating metabolic disease comprising the following steps:
1) treating the cells expressing the fusion protein of claim 1 and having an increased intracellular calcium concentration with a test substance;
2) measuring the binding between the fusion protein of claim 1 and calcium in the cells of step 1); and
3) selecting a test substance that can inhibit the binding between the fusion protein of claim 1 and calcium in step 2).

13. The method for screening a candidate substance for treating metabolic disease according to claim 12, wherein the calcium is bound to phosphatidylinositol (3,4,5)-triphosphate or phosphatidylinositol (4,5)-bisphosphate.

14. The method for screening a candidate substance for treating metabolic disease according to claim 12, wherein the binding in step 2) is measured by one or more methods selected from the group consisting of ITC (isothermal titration calorimetry) and protein-lipid binding assay.

15. The method for screening a candidate substance for treating metabolic disease according to claim 12, wherein the cells showing an increased intracellular calcium concentration therein are the model cells of obesity, insulin resistance, diabetes or fatty liver.

16. The method for screening a candidate substance for treating metabolic disease according to claim 12, wherein the metabolic disease is obesity, insulin resistance, diabetes or fatty liver.

17. The method for screening a candidate substance for treating metabolic disease according to claim 12, wherein the method includes an additional step of selecting a material that can inhibit the migration of the fusion protein of claim 1 to the cell membrane.

18. The method for screening a candidate substance for treating metabolic disease according to claim 12, wherein the method includes an additional step of selecting a test substance that can inhibit the Akt signal transduction by the fusion protein of claim 1.

19. A method for screening a candidate substance for treating metabolic disease comprising the following steps:
1) treating the mixture consisting of the fusion protein of claim 1 and a calcium and PIP complex with a test substance in vitro;
2) measuring the binding between the fusion protein of claim 1 and the calcium and PIP complex in the mixture of step 1); and
selecting a test substance that can inhibit the conjugation between the fusion protein of claim 1 and calcium in step 2).


SEQUENCE LISTING (17 sequences)

SEQ ID NO: 1 - DNA, 35 bp, Artificial
  artificial_hPKC beta1_C2_PN127(NcoI)
aaaccatgggccaccaggggatgaaatgtgacacc

SEQ ID NO: 2 - DNA, 63 bp, Artificial
  artificial_hPKC beta1_C2_PC296(XhoI)
aaactcgagcagatcctcttctgagatgagtttttgttcctcacttccttctggtggcacagg

SEQ ID NO: 3 - DNA, 29 bp, Artificial
  artificial_hAkt1_PH_PN1(SacI)
aaagagctcatgggcagcgacgtggctat

SEQ ID NO: 4 - DNA, 50 bp, Artificial
  artificial_hAKT1_PH_PC144(SpeI)
aaaactagttcagtggtggtggtggtggtggcggtgcttgggcttggcca

SEQ ID NO: 5 - DNA, 32 bp, Artificial
  artificial_hPKC beta1_C2_PN159(BglII)
aaaagatctatgcgcggccgcatctacatcca

SEQ ID NO: 6 - DNA, 29 bp, Artificial
  artificial_hPKC beta1_C2_PC289(BanHI)
aaaggatccaggcacattgaagtactcgc

SEQ ID NO: 7 - DNA, 30 bp, Artificial
  artificial_hAkt1_PH_PN6(BglII)
aaaagatctatgattgtgaaggagggttgg

SEQ ID NO: 8 - DNA, 27 bp, Artificial
  artificial_hAkt1_PH_PN111(BamHI)
aaaggatcccttgaggccgtcagccac

SEQ ID NO: 9 - DNA, 31 bp, Artificial
  artificial_hPKC beta1_C2_PN127(BglII)
aaaagatctcgctcattgtcctcgtaagaga

SEQ ID NO: 10 - DNA, 29 bp, Artificial
  artificial_hPKC beta1_C2_PC296(NheI)
aaagctagcggacaaagatcccatgaagt

SEQ ID NO: 11 - DNA, 29 bp, Artificial
  artificial_hAKT1_kinase_PN(NheI)
aaagctagctttgagtacctgaagctgct

SEQ ID NO: 12 - DNA, 29 bp, Artificial
  artificial_hAKT1_kinase_PC(KpnI)
aaaggtacctcaggccgtgccgctggccg

SEQ ID NO: 13 - protein, 144 aa, Artificial
  artificial_PH domain of Akt protein
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHR

SEQ ID NO: 14 - protein, 124 aa, Artificial
  artificial_C2 domain of PKC beta protein
HQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLT

SEQ ID NO: 15 - protein, 370 aa, Artificial
  artificial_Akt protein w/o PH domain
KKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASGTA

SEQ ID NO: 16 - protein, 480 aa, Artificial [sequence omitted]

SEQ ID NO: 17 - protein, 667 aa, Artificial
  artificial_C2-Akt fusion protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGRTQISLIVLVRDAKNLVPMDPNGLSDPYVKLKLIPDPKSESKQKTKTIKCSLNPEWNETFRFQLKESDKDRRLSVEIWDWDLTSRNDFMGSLSASFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASGTA